(19) United States
(12) Patent Application Publication
(10) Pub. No.: US 2004/0224377 A1
(43) Pub. Date: Nov. 11, 2004

(54) Compositions and methods to quench light from optical reactions

(76) Inventors: Erika Hawkins, Madison, WI (US); Braeden Butler, Madison, WI (US); Keith V. Wood, Mt. Horeb, WI (US)

Correspondence Address: Schwegman, Lundberg, Woessner & Kluth, P.A., P.O. Box 2938, Minneapolis, MN, 55402, US

(21) Appl. No.: 10/777,461

(22) Filed: Feb. 12, 2004

Related U.S. Application Data
(60) Provisional application No. 60/447,065, filed on Feb. 12, 2003.

(51) Int. Cl. C12Q 1/66

(52) U.S. Cl. 435/8

(57) ABSTRACT

The present invention relates to single and dual reporter luminescence assays utilizing reagents to quench an optical, e.g., an enzyme-mediated luminescence, reaction. In one embodiment of the invention, a reagent is added to an assay which selectively quenches a first enzyme-mediated luminescence reaction without affecting a subsequent distinct enzyme-mediated luminescent reaction(s). An assay kit containing one or more selective quench reagents, and compositions comprising the quench reagent(s), are also provided.

### CROSS-REFERENCE TO RELATED APPLICATIONS

This application claims the benefit of the filing date of U.S. application Ser. No. 60/447,065, filed on Feb. 12, 2003, the disclosure of which is incorporated by reference herein.

### FIELD OF THE INVENTION

The present invention relates to enzyme-mediated single and dual optical reporter assays, and reagents which quench one or more optical reactions. For example, the present invention relates to luminescence assays utilizing at least one enzyme, and one or more luminescence quench reagents.

### BACKGROUND OF THE INVENTION

Luminescence is produced in certain organisms as a result of a luciferase-mediated oxidation reaction. Luciferase genes from a wide variety of vastly different species, particularly the luciferase genes of Photinus pyralis (the common firefly of North America), Pyrophorus plagiophthalamus (the Jamaican click beetle), Renilla reniformis (the sea pansy), and several bacteria (e.g., Xenorhabdus luminescens and Vibrio spp), are extremely popular luminescence reporter genes. Firefly luciferase is also a popular reporter for ATP concentrations, and, in that role, is widely used to detect biomass. Luminescence is also produced by other enzymes when those enzymes are mixed with certain synthetic substrates, for instance, alkaline phosphatase and adamantyl dioxetanes, or horseradish peroxidase and luminol.

Luciferase genes are widely used as genetic reporters due to the non-radioactive nature, sensitivity, and extreme linear range of luminescence assays. For instance, as few as 10⁻²⁰ moles of firefly luciferase can be detected. Consequently, luciferase assays of gene activity are used in virtually every experimental biological system, including both prokaryotic and eukaryotic cell cultures, transgenic plants and animals, and cell-free expression systems. Similarly, luciferase assays of ATP are highly sensitive, enabling detection to below 10⁻¹⁶ moles.

Luciferases generate light via the oxidation of enzyme-specific substrates, called luciferins. For firefly luciferase and all other beetle luciferases, light generation occurs in the presence of magnesium ions, oxygen, and ATP. For anthozoan luciferases, including Renilla luciferase, only oxygen is required along with the luciferin. Generally, in luminescence assays of genetic activity, reaction substrates and other luminescence activating reagents are introduced into a biological system suspected of expressing a reporter enzyme. Resultant luminescence, if any, is then measured using a luminometer or any suitable radiant energy-measuring device. The assay is very rapid and sensitive, and provides gene expression data quickly and easily, without the need for radioactive reagents. Reporter assays other than for genetic activity are performed analogously.

The conventional assay of genetic activity using firefly luciferase has been further improved by including coenzyme A (CoA) in the assay reagent to yield greater enzyme turnover and thus greater luminescence intensity (U.S. Pat. No. 5,283,179). Using this reagent, luciferase activity can be readily measured in luminometers or scintillation counters. The luciferase reaction, modified by the addition of CoA to produce persistent light emission, provides an extremely sensitive and rapid assay for quantifying luciferase expression in genetically altered cells or tissues.

Light refracted from one luminous sample may interfere with the subsequent measurement of signal from luminescent samples in successive wells in clear multi-wells. Moreover, with respect to the cumulative nature of refracted light emanating from multiple luminous samples within a single clear plastic plate, while the luminescent signal in the first sample well could be measured accurately, sequential activation of luminescent reactions in following wells would lead to increasingly inaccurate measurements due to the cumulative emission of photons refracted through the plastic from all of the previous samples. This problem of refracted light, or “refractive cross-talk”, would be further exacerbated when brightly illuminated wells were situated adjacent to negative control wells in which no luminescence was generated, or when brightly lit wells were situated near relatively dim wells. This makes determining the absolute and baseline luminescence in a clear multi-well plate quite difficult.

Opaque plates formed of white plastic can yield greater luminescence sensitivity than clear plates, however, photons are readily scattered from adjacent wells, again introducing cross-talk interference between wells. Here, the cross-talk is referred to as “reflective cross-talk.” Moreover, black 96-well plates, originally intended for fluorescent applications, are not ideal for luminescence applications because the sample signal is greatly diminished due to the non-reflective nature of the plastic. Further, opaque plates are inferior for cultured cells because cultured cells cannot be viewed or photographed through the opaque plate, and the plates have undetermined effects on cell adhesion and growth characteristics of the cells.

Luciferases are one of a number of reporters, e.g., firefly luciferase, Renilla luciferase, chloramphenicol acetyl transferase (CAT), beta-galactosidase (lacZ), beta-glucuronidase (GUS) and various phosphatases, such as secreted alkaline phosphatase (SEAP) and uteroferrin (Uf; an acid phosphatase), that have been combined and used as co-reporters of genetic activity. A dual enzyme reporter system relates to the simultaneous use, expression, and measurement of two individual reporter enzymes within a single system. In genetic reporting, dual reporter assays are particularly useful for assays in individual cells or cell populations (such as cells dispersed in culture, segregated tissues, or whole animals) genetically manipulated to simultaneously express two different reporter genes. Most frequently, the activity of one gene reports the impact of the specific experimental conditions, while the activity of the second reporter gene provides an internal control by which all sets of experimental values can be normalized. Dual enzyme reporter technology can also be employed with cell-free reconstituted systems such as cellular lysates derived for the simultaneous translation, or coupled transcription and translation, of independent genetic materials encoding experimental and control reporter enzymes. Immunoassays may, likewise, be designed for dual reporting of both experimental and control values from within a single sample.

The performance of any dual enzyme reporter assay is limited by the characteristics of the constituent enzyme chemistries and the ability to correlate their respective resulting data sets. Disparate enzyme kinetics, assay chemistries and incubation requirements of various reporter enzymes can complicate combining two reporter enzymes into an integrated, single tube or well dual reporter assay format. One approach to integration for a dual reporter assay is described in U.S. Pat. No. 5,744,320, which discloses particular general or specific quenching agents for beetle or Renilla luciferase assays and demonstrates an exemplary dual reporter assay for sequentially determining luminescence from firefly luciferase then Renilla luciferase.

However, what is needed is the identification of further luminescence quench agents for use in a method to assay an enzyme-mediated luminescence reaction or a series of enzyme-mediated luminescence reactions.

### SUMMARY OF THE INVENTION

The present invention is directed to compositions and methods to quench (reduce, inhibit or eliminate) light generated by one luminescent reporter so that a second luminescent reporter signal may be subsequently measured. Such a method provides for multiplexing various combinations of light producing reactions with great flexibility and without regard to the nature of the light producing reaction or to the sequence in which the reactions are measured. Thus, the invention includes compositions and methods for luminescence assays which utilize one or more reagents to rapidly and efficiently quench, e.g., selectively quench, a first enzyme-mediated luminescence reaction. Preferred selective quenching reagents for use in the methods and compositions of the invention include, but are not limited to, a substrate analog inhibitor for a first enzyme, e.g., one that is structurally similar to a native substrate for the enzyme (i.e., a substrate for the enzyme which occurs in nature) and inhibits the enzyme and/or one that competes with a light generating substrate for the active site on an enzyme (a competitive inhibitor); a sequestering agent, e.g., an agent which physically separates a substrate for a first enzyme from the first enzyme, for instance, the agent physically separates the first substrate or first enzyme into micelles or shifts the solubility of the first substrate or the first enzyme, so as to inhibit an interaction between the first substrate and the first enzyme which results in light generation but does not substantially alter a reaction between a second, distinct enzyme and its corresponding substrate; a colored compound, which quenches the color of light emitted by at least one enzyme-mediated luminescence reaction but not all enzyme-mediated reactions, and including other suitable organic compounds; which substantially quench one enzyme-mediated luminescence reaction but not all enzyme-mediated luminescence reactions to the same degree, or any combination thereof. Thus, such reagents are selective in that, in an effective amount, they quench at least one enzyme-mediated luminescence reaction while permitting efficient generation and recordation of light from at least one other distinct enzyme-mediated luminescence reaction. In one embodiment, selective quenching reagents for a first enzyme-mediated luminescence reaction are not reagents that selectively quench luminescence from a beetle luciferase-mediated reaction. Preferably, one or more selective quenching reagents for a first enzyme-mediated luminescence reaction are reagents that selectively quench luminescence from an anthozoan luciferase-mediated reaction.

A “substantial” quenching of light is a fold-quench equal to or greater than the fold quench for a reference, e.g., a first enzyme-mediated luminescence reaction. For instance, a selective quench reagent would substantially quench a first enzyme-mediated luminescence reaction by 35-fold, but would not quench or quenches a second, distinct enzyme-mediated luminescence reaction by less than 35-fold, therefore, it is a selective quench reagent for the first reaction relative to the second reaction. In contrast, if a quench reagent quenches a first enzyme-mediated luminescence reaction by 35-fold and quenches a second, distinct enzyme-mediated luminescence reaction by 35-fold or more, it is not a selective quench reagent for the first reaction relative to the second reaction.

A selective quench reagent would quench luminescence from a luminescent reaction by at least 15-fold, preferably by at least 25-fold, more preferably by at least 35-fold, and even more preferably by at least 50-fold, and yet even more preferably by at least 100-fold or more, e.g., 200-fold, 300-fold, 400-fold, or 900-fold, when compared to a distinct luminescent reaction.

A luminescence reporter is a molecule which mediates a luminescence reaction, and by doing so, yields information about the state of a chemical or biochemical system. Examples are genetic reporters (Wood, 1995), immunoassay reporters (Bronstein et al., 1991), ATP reporters (Schram, 1991), as well as reporters of other cellular molecules such as enzymes or cofactors. Enzymes are proteins which catalyze a chemical transformation, and thus are not changed by that transformation. Because the enzyme is regenerated at the conclusion of the transformation, it is available for additional cycles of transformation; enzymes thus have the capacity for substrate turnover. This property allows the capacity for continuous luminescence in an enzyme-mediated luminescence reaction. An enzyme-mediated luminescence reaction is a chemical reaction mediated by an enzyme which yields photons as a consequence of the reaction. The enzyme in an enzyme-mediated luminescence reaction effectively enables the reaction when the majority of the luminescence generated in the reaction follows as a consequence of the action of the enzyme.

The present invention is ideally suited for luminescence reactions as photons are transient in existence. Therefore, quenching of an enzymatic reaction which produces photons immediately diminishes the product photons present in the sample. Thus, once the luminescence measurement is taken, and the enzymatic reaction is quenched, there is no build-up of product photons in the sample. In essence, luminescence reactions can be “turned off” without leaving an accumulation of the experimental or control signal (i.e., photons) within the sample. The same cannot be said of analogous enzymatic reactions in which the buildup of a stable chemical product is measured, or the slow decay of an accumulated chemical product is measured. Here, quenching enzymatic reactions leading to a chemical product still leaves a large accumulation of the chemical product within the sample, leading to potential interference with other assays being simultaneously or sequentially taken from the sample.

Examples of enzymes which mediate luminescence reactions include, but are not limited to, beetle luciferases, which all catalyze ATP-mediated oxidation of beetle luciferin; anthozoan luciferases, which all catalyze oxidation of coelenterazine (Ward, 1985); a peroxidase such as horseradish peroxidase, which catalyzes a reaction involving luminol (Thorp et al., 1986); and a phosphatase such as alkaline phosphatase, which catalyzes a reaction with adamantyl 1,2-dioxetane phosphate (Schaap et al., 1989), as well as other enzymes which catalyze a reaction with a dioxetane substrate, e.g., a substrate such as 3-(2′-spiroadamantane)-4-methoxy-4-(3″-phosphoryloxy)phenyl-1,2-dioxetane, disodium salt, or disodium 3-(4-methoxyspiro[1,2-dioxetane-3,2′(5′-chloro)-tricyclo-[3.3.1.1^{3,7}]decan]-4-yl]phenyl phosphate, or disodium 2-chloro-5-(4-methoxyspiro{1,2-dioxetane-3,2′-(⁵′-chloro)-tricyclo {3.3.1.13,7]decan}-4-yl)-1-phenyl phosphate, disodium 2-chloro-5-(⁴-methoxyspiro{1,2-dioxetane-3,2′-tricyclo[3.3.1.13,7]decan}-4-yl)-1-phenzyl phosphate (AMPPD, CSPD, CDP-Star® and ADP-Star™, respectively), 3-(2′-spiroadamantane)-4-methoxy-4-(3″-β-D-galactopyranosyl)phenyl-1,2-dioxetane (AMPGD), 3-(4-methoxyspiro[1,2-dioxetane-3,2′-(5′-chloro)tricyclo[3.3.1.1^{3,7}]-decan]-4-yl-phenyl-β-D-galactopyranoside (Galacton®), 5-chloro-3-(methoxyspiro[1,2-dioxetane-3,2′-(5′-chloro)tricyclo[3.3.1^{3,7}]decan-4-yl-phenyl-β-D-galactopyranoside (Galacton-Plus®), 2-chloro-5-(4-methoxyspiro[1,2-dioxetane-3,2′(5′-chloro)-tricyclo-[3.3.1.1^{3,7}]decan]-4-yl)phenyl β-D-galactopyranoside (Galacton-Star®), and sodium 3-(4-methoxyspiro{1,2-dioxetane-3,2′-(5′-chloro)-tricyclo[3.3.1.1^{3,7}]decan}-4-yl)phenyl-β-D-glucuronate (Glucuron™); or a functional equivalent of such an enzyme. A functional equivalent of a specified enzyme includes a recombinant enzyme that maintains the ability to catalyze the same luminescence reaction as the corresponding nonrecombinant wild-type enzyme, and thus it remains in the same group of enzymes, but has an altered structure relative to a corresponding wild-type enzyme. An example of a functional equivalent of an enzyme is a genetic fusion of one enzyme to another peptide or protein to yield a bifunctional hybrid protein (Kobatake et al., 1993).

Luciferases can be isolated or obtained from a variety of luminous organisms, such as the firefly luciferase of Photinus pyralis or the Renilla luciferase of Renilla reniformis. A “luciferase” as used herein shall mean any type of luciferase originating from any natural, synthetic, or genetically-altered source, including, but not limited to: luciferases isolated from the firefly Photinus pyralis or other beetle luciferases (such as luciferases obtained from click beetles (e.g., Pyrophorus plagiophthalamus) or glow worms (Pheogodidae spp.)), the sea pansy Renilla reniformis, Vargula species, e.g., Vargula hilgendorfii, Gaussia species, Oplophorus species, the limpet Latia neritoides, and bacterial luciferases isolated from such organisms as Xenorhabdus luminescens, and Vibrio fisherii; and functional equivalents thereof.

In one embodiment, the present invention relates to luminescence assays which employ one or more reagents which quench an enzyme-mediated luminescence reaction. In one embodiment, the one or more quench reagent(s) are added in an amount effective to quench luminescence by at least 15-fold, preferably by at least 25-fold, more preferably by at least 35-fold, and even more preferably by at least 50-fold, and yet even more preferably by at least 100-fold or more, e.g., 200-fold, 300-fold, 400-fold, or 900-fold, relative to the luminescence generated in the absence of the reagent(s). Preferably, the quench reagent is a selective quench reagent as described herein.

For example, the invention includes a method of assaying an enzyme-mediated luminescence reaction. The method includes detecting or determining luminescence energy produced by at least one first enzyme-mediated luminescence reaction and quenching photon emission from the first enzyme-mediated luminescence reaction by introducing a composition comprising at least one selective quench reagent to the luminescence reaction. In another embodiment, the method includes detecting or determining luminescence energy produced by at least one first enzyme-mediated luminescence reaction and quenching photon emission from the first enzyme-mediated luminescence reaction by introducing a composition comprising at least two selective quench reagents to the luminescence reaction.

In another embodiment, the present invention relates to luminescence assays which employ one or more reagents which selectively quench a first enzyme-mediated luminescence reaction without substantially quenching the light generated by a second distinct, sequential enzyme-mediated luminescence reaction. In one embodiment, at least one reagent for the second distinct, enzyme-mediated luminescence reaction is present in the first enzyme-mediated luminescence reaction.

In one embodiment of the invention, an enzyme-mediated luminescence reaction is first initiated by addition of an appropriate initiating reagent or reagents to a sample to yield a reaction mixture. The luminescence signal produced in the reaction mixture is then measured, e.g., so as to detect the presence or amount of one or more molecules in the sample. One or more selective quench reagents are then added so as to diminish the luminescence signal within a relatively short time interval after introduction of the selective quench reagent. In one embodiment, the one or more selective quench reagent(s) are added in an amount effective to quench luminescence by at least 15-fold, preferably by at least 25-fold, more preferably by at least 35-fold, and even more preferably by at least 50-fold, and yet even more preferably by at least 100-fold or more, e.g., 200-fold, 300-fold, 400-fold, or 900-fold, relative to the luminescence generated in the absence of the reagent(s). By extinguishing the luminescence signal from the enzyme in the sample, addition of the selective quench reagent(s) prevents light from previously-activated samples from interfering with light measurements in subsequently-activated samples, e.g., in a multisample assay format. The second luminescence signal produced is then measured. Preferably, the presence or amount of two or more molecules are detected in a single reaction, e.g., all reactions are conducted in a single receptacle, e.g., well.

The sample employed in the methods of the invention may be a cell lysate, an in vitro transcription/translation reaction, a supernatant of a cell culture, a physiological fluid sample, e.g., a blood, plasma, serum, cerebrospinal fluid, tears or urine sample, and may include intact cells. The cells, cell lysate, or supernatant may be obtained from prokaryotic cells or eukaryotic cells, and the physiological fluid from any avian, reptile, amphibian or mammal. The initiating reagent or reagents may thus be added to intact cells, cell lysates, or supernatants or physiological fluids. The quench reagent may also be added to intact cells, or to a cell lysate, an in vitro transcription/translation reaction, or a physiological fluid sample or supernatant sample.

The present invention thus includes dual reporter luminescence assays which employ one or more reagents which selectively quench one enzyme-mediated luminescence reaction, e.g., a luciferase-mediated luminescence reaction or a non-luciferase mediated luminescence reaction, without quenching another distinct enzyme-mediated luminescence reaction, i.e., the two distinct enzymes respond differently to various reagents, thereby allowing one of the enzyme-mediated luminescence reactions to be selectively quenched. In one embodiment, both reactions are luciferase-mediated reactions, e.g., the first luciferase-mediated luminescence reaction is a Renilla luciferase-mediated luminescence reaction, which is selectively quenched while allowing a second distinct luciferase-mediated luminescence reaction, for instance, a firefly luciferase-mediated luminescence reaction, to proceed without substantially quenching the luminescence from the second reaction. For example, Renilla luciferase can be selectively quenched using reagents which are selective for anthozoan luciferases and have no effect on other reporters present in or reactions occurring in the sample. Exemplary reagents for selectively quenching anthozoan luciferase-mediated luminescence reactions include, but are not limited to, a substrate analog inhibitor which is structurally related to coelenterazine, a detergent, e.g., one which sequesters an anthozoan luciferase substrate but not the anthozoan luciferase enzyme in micelles, a colored compound which selectively quenches the color emitted by the first reaction, for instance, for blue light, a selective quench reagent is a yellow colored compound, or a combination of such reagents.

The quench reagent for the first reaction and the activation reagent for the second reaction can be added simultaneously or sequentially. When the quench reagent is formulated to allow simultaneous initiation of a second enzyme-mediated luminescence reaction, the reagent is referred to as a “quench-and-activate” reagent. Hence, a quench-and-activate reagent simultaneously quenches the first enzymatic reaction and initiates the second enzymatic reaction and such an assay thus allows the sequential measurement of two separate and distinct luminescence reporters within one sample. As a result, one of the luminescence reporters can be used as an internal standard, while the other is used to report the impact of the experimental variables. Alternatively, each reporter can report two different variables, e.g., the presence of a particular protease and ATP concentration, in a sample. This strategy greatly expedites multiplexing to provide quick, automatable, accurate, and reproducible results using standard multi-well plates and instrumentation.

For instance, the luminescence chemistries of beta-galactosidase, beta-glucuronidase, horseradish peroxidase, alkaline phosphatase or luciferases can be utilized in a dual reporter luminescence assay with a distinct luminescence enzyme. In one embodiment, one of the two luminescent enzymes acts as an internal standard, while the other functions as an experimental marker for gene activity or the presence or amount of an enzyme, substrate or cofactor for an enzyme-mediated reaction. Moreover, the present invention is particularly useful for high-throughput automated assays based on enzyme-mediated luminescence reporter systems, using conventional transparent or opaque multi-well plates.

In one embodiment, the invention includes a method of assaying an enzyme-mediated luminescence reaction. The method includes detecting or determining luminescence energy produced by at least one first enzyme-mediated luminescence reaction; and quenching photon emission from the first enzyme-mediated luminescence reaction and/or quenching the first enzyme-mediated luminescence reaction by introducing at least one quench reagent to the luminescence reaction. In one embodiment, the quench reagent is a substrate analog inhibitor of an anthozoan luciferase, a colored compound, a sequestering agent, or a combination thereof. For instance, in one embodiment, an anthozoan luciferase-mediated luminescence reaction may be employed to detect the presence or amount of a molecule, e.g., a protease, which reaction is quenched prior to initiating a beetle luciferase-mediated luminescence reaction, e.g., to detect ATP concentration. Accordingly, the present invention allows multiplexing of enzyme-mediated assays for one or more enzymes, one or more substrates and/or one or more cofactors, or any combination thereof.

The invention thus provides a method for measuring the activity or presence of at least one molecule in a sample. The method includes providing a sample that may contain at least one molecule for an enzyme-mediated reaction, e.g., the sample may contain the enzyme, and contacting the sample with a reaction mixture for the enzyme-mediated reaction which lacks the molecule, e.g., the reaction mixture contains a substrate for the enzyme to be detected, where the presence or amount of the molecule is capable of being detected by an enzyme-mediated luminescence reaction. In one embodiment, after or concurrently with quenching the first enzyme-mediated luminescence reaction, the reaction mixture is contacted with reagents to detect a molecule capable of being detected by a second enzyme-mediated luminescence reaction.

The methods of the present invention allow the detection of multiple enzymes, substrates or cofactors in a sample, e.g., a sample which includes eukaryotic cells, e.g., yeast, avian, plant, insect or mammalian cells, including but not limited to human, simian, murine, canine, bovine, equine, feline, ovine, caprine or swine cells, or prokaryotic cells, or cells from two or more different organisms, or cell lysates or supernatants thereof. The cells may not have been genetically modified via recombinant techniques (nonrecombinant cells), or may be recombinant cells which are transiently transfected with recombinant DNA and/or the genome of which is stably augmented with a recombinant DNA, or which genome has been modified to disrupt a gene, e.g., disrupt a promoter, intron or open reading frame, or replace one DNA fragment with another. The recombinant DNA or replacement DNA fragment may encode a molecule to be detected by the methods of the invention, a moiety which alters the level or activity of the molecule to be detected, and/or a gene product unrelated to the molecule or moiety that alters the level or activity of the molecule.

In one embodiment, the present invention relates to a method of measuring the presence or amount of multiple enzymes in a single aliquot of cells or a lysate thereof. For enzymes present in different cellular locations, such as a secreted and an intracellular enzyme, a substrate for one of the enzymes can be added to a well with intact cells. Thus, in one embodiment, the presence or amount of the secreted enzyme is detected by contacting intact cells with reagents for an enzyme-mediated luminescence reaction and a substrate for the secreted enzyme, which substrate, when cleaved, yields a substrate for the luminescence reaction, then a selective quench reagent is added concurrently with, before or after cells are lysed, and the presence or amount of the intracellular enzyme is detected, e.g., where the detection of the intracellular enzyme is in the same receptacle, for instance, same well, as that for the secreted enzyme. Detection of the first enzyme may be before cell lysis or after cell lysis but before quenching. Thus, the present methods can be employed to detect any molecule in an enzyme-mediated reaction including any enzyme, substrate or cofactor, or any set thereof. Enzymes employed in the methods, either enzymes to be detected or enzymes which are useful to detect a substrate or cofactor, can be selected from any combination of enzymes including recombinant and endogenous (native) enzymes.

The invention also includes quench reagents, compositions and assay kits for analyzing samples using enzyme-mediated luminescence reactions. For example, the invention includes an enzyme-mediated luminescence reaction assay kit which includes at least one functional enzyme substrate corresponding to the enzyme-mediated luminescence reaction to be assayed; a suitable first container, the at least one functional enzyme substrate disposed therein; a composition comprising at least one selective quench reagent, wherein at least one of the selective quench reagents comprises a substrate analog inhibitor for the enzyme, a colored compound, a sequestering agent, or other organic compound; a suitable second container, the composition disposed therein; and instructions for use. The functional enzyme substrates may be obtained from organisms (“native” substrates) or prepared in vitro (“synthetic” substrates). In another embodiment, the enzyme-mediated luminescence reaction assay kit includes at least one functional enzyme substrate corresponding to the enzyme-mediated luminescence reaction to be assayed; a suitable first container, the at least one functional enzyme substrate disposed therein; a composition comprising at least one selective quench reagent for an anthozoan luciferase; a suitable second container, the composition disposed therein; and instructions for use. Kits may also include control reagents, e.g., functional enzymes.

In another embodiment, the invention includes a dual reporter enzyme-mediated luminescence reaction assay kit which includes a first functional enzyme substrate corresponding to a first enzyme-mediated luminescence reaction being assayed; a suitable first container, the first functional enzyme substrate disposed therein; a quench-and-activate composition which includes at least one selective quench reagent for an anthozoan luciferase and a second and distinct functional enzyme substrate corresponding to a second and distinct enzyme-mediated luminescence reaction; a suitable second container, the quench-and-activate composition disposed therein; and instructions for use. In yet another embodiment, the dual reporter enzyme-mediated luminescence reaction assay kit includes a first functional enzyme substrate corresponding to a first enzyme-mediated luminescence reaction being assayed; a suitable first container, the first functional enzyme substrate disposed therein; a quench-and-activate composition comprising at least two selective quench reagents and a second and distinct functional enzyme substrate corresponding to a second and distinct enzyme-mediated luminescence reaction; a suitable second container, the quench-and-activate composition disposed therein; and instructions for use.

Also provided is a method to reduce or inhibit analyte-independent or analyte-dependent phosphorescence in an enzyme-mediated luminescence reaction. An “analyte” as used herein is a substance present in a luminescence reaction mixture which produces phosphorescence. An example of a “non-analyte” is a substance which is not present in a luminescence reaction mixture such as a vessel or receptacle, e.g., a white luminometer plate, which produces phosphorescence in the absence of an analyte. “Phosphorescence” is the gradual release of energy over time in the visible band from phosphors that have absorbed high energy electrons directed at them. In contrast, fluorescence is the radiation of energy of one frequency from particles that have absorbed high energy electrons of a different frequency. The method comprises contacting a sample comprising an enzyme that mediates a luminescence reaction with a reaction mixture for the enzyme, which mixture comprises a colored compound but does not comprise the enzyme for the luminescent reaction. The color of the compound is substantially the same, i.e., within about 75 nm, preferably within about 50 nm, and more preferably within 25 nm, 10 nm, or less, e.g., within 5 mm, as the light emitted by the luminescence reaction. Then luminescence is detected or determined. Also provided is a method for identifying a compound useful to reduce or inhibit analyte-independent or analyte-dependent phosphorescence in an enzyme-mediated luminescence reaction. The method comprises contacting one or more compounds with a reaction mixture comprising an enzyme that mediates a luminescence reaction and identifying a compound that reduces or inhibits analyte-independent or analyte-dependent phosphorescence in the enzyme-mediated luminescence reaction.

In one embodiment, the invention includes an enzyme-mediated luminescence reaction assay kit which includes at least one functional enzyme substrate for the enzyme-mediated luminescence reaction to be assayed; a suitable first container, the at least one functional enzyme substrate disposed therein; at least one colored compound; a suitable second container, the at least one colored compound disposed therein; and instructions for use, wherein the color of the at least one compound is substantially the same as the light emitted by the enzyme-mediated luminescence reaction. In one embodiment, an enzyme-mediated luminescence reaction assay kit to reduce or inhibit analyte-independent or analyte-dependent phosphorescence is provided. The kit comprises at least one colored compound; a suitable first container for the at least one colored compound; at least one functional enzyme substrate corresponding to the enzyme-mediated luminescence reaction to be assayed; a suitable second container, the at least one functional enzyme substrate disposed therein; and instructions for use. In another embodiment, the kit comprises at least one colored compound and at least one functional enzyme substrate corresponding to the enzyme-mediated luminescence reaction to be assayed, a suitable container for the colored compound and the at least one functional enzyme substrate, and instructions for use. The color of the compound in the kit is substantially the same as the light emitted by the enzyme in the luminescent reaction.

### BRIEF DESCRIPTION OF THE FIGURES

FIG. 1 shows that firefly luciferase luminescence is not affected by a substrate analog of Renilla luciferase, e.g., coelenterazine hh methyl ether.

FIG. 2 illustrates that firefly luciferase luminescence increases in the presence of a sequestering agent of the invention.

FIG. 3 shows that firefly luciferase luminescence is not affected by a yellow colored compound, e.g., berberine hemisulfate.

FIG. 4 shows that a yellow compound, the dye berberine hemisulfate, quenches horseradish peroxidase chemiluminescence.

FIG. 5 illustrates properties of selected detergents.

### DETAILED DESCRIPTION OF THE INVENTION

The present invention includes a method of assaying enzyme-mediated luminescence reactions. In one embodiment, the method includes initiating at least one first enzyme-mediated luminescence reaction, quantifying luminescence energy produced by the luminescence reaction, and quenching photon emission from the first enzyme-mediated luminescence reaction by introducing a composition comprising at least one quench reagent to the luminescence reaction. Preferably, the quench reagent is a selective quench reagent, i.e., the quench reagent does not quench all luminescence reactions and so a second sequential enzyme-mediated luminescence reaction may be conducted. Thus, the invention provides compositions and methods useful to quench as well as selectively quench a first enzyme-mediated luminescence reaction.

The present invention also includes a dual reporter method for assaying enzyme-mediated luminescence reactions in which a first enzyme-mediated luminescence reaction is initiated, and the luminescent energy of the first reaction detected or determined. This is followed by introduction of a composition comprising at least one selective quench reagent, i.e., a quench reagent which quenches at least one but not all luminescence reactions, then by a composition comprising a mixture capable of activating or initiating the second enzyme-mediated luminescence reaction, or by a quench-and-activate composition capable of selectively quenching the first enzyme-mediated luminescence reaction and simultaneously initiating a second enzyme-mediated luminescence reaction which is distinct from the first enzyme-mediated luminescence reaction. The luminescent energy produced by the second enzyme-mediated luminescence reaction is then detected or determined. Optionally, the second enzyme-mediated luminescence reaction may subsequently be quenched by the addition of a second quench reagent, which may be selective for the second enzyme-mediated luminescence reaction and preferably does not quench or does not substantially quench a third enzyme-mediated luminescence reaction.

The selective quench reagents are ideally suited for use with automatic injectors and in microtiter plates (both opaque and clear) such as conventional 96-well plates. Because the selective quench reagent effectively extinguishes the luminescence signal from within a sample, multiple luminescence assays can be performed within a clear multi-well plate without refractive cross-talk between samples. Moreover, the selective quench reagent eliminates unacceptable levels of reflected background light.

In one preferred embodiment, at least one of the enzyme-mediated luminescence reactions is a luciferase-mediated reaction. Among luciferases specifically, the method of the present invention may be used to assay luminescence reactions mediated by anthozoan luciferases including Renilla reniformis luciferase, as well as beetle luciferases, including Photinus pyralis luciferase, and Pyrophorus plagiophthalamus luciferase. For instance, the first enzyme-mediated luminescence reaction may be an anthozoan luciferase-mediated reaction. In another embodiment, the first luciferase-mediated luminescence reaction is not mediated by a beetle luciferase, e.g., a firefly luciferase. In one embodiment, the first luciferase-mediated luminescence reaction is mediated by Renilla luciferase and the second enzyme-mediated reaction is mediated by a distinct enzyme such as beetle luciferase, horseradish peroxidase, alkaline phosphatase, beta-glucuronidase or beta-galactosidase. In another embodiment, the first enzyme-mediated luminescence reaction may be mediated by an enzyme which is not a luciferase such as a peroxidase or a phosphatase. In this embodiment, the second enzyme-mediated reaction may be mediated by an enzyme such as a luciferase, beta-glucuronidase or beta-galactosidase.

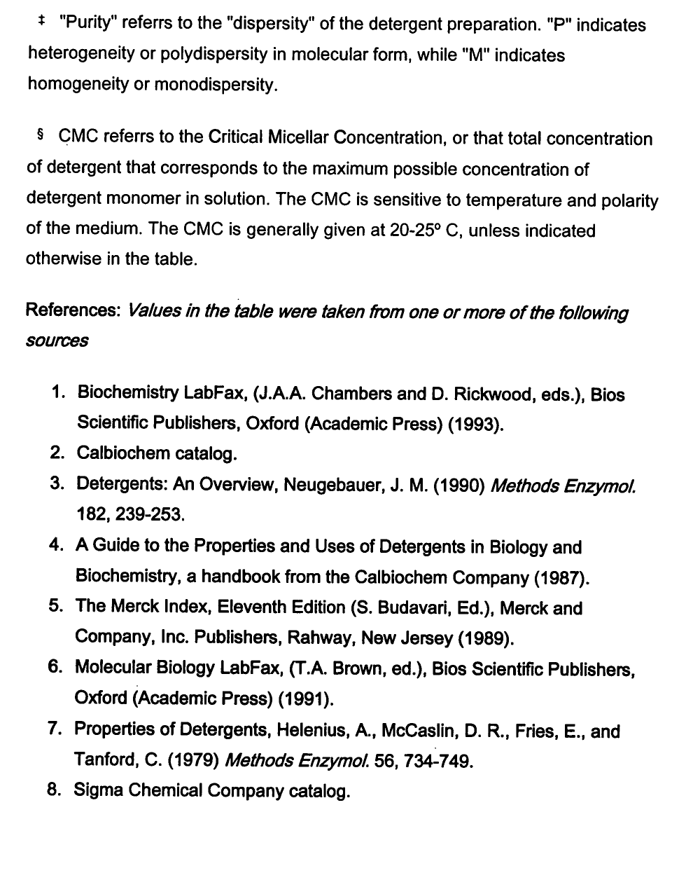

As described herein, an enzyme-mediated luminescence reaction may be quenched and preferably selectively quenched with a number of different reagents including, but not limited to, one or more quench reagents selected from the following classes of compounds: a substrate analog inhibitor for a luminescence reaction, a sequestering agent such as a compound which can physically separate the enzyme from its substrate, e.g., a detergent, or compound which otherwise alters solubility of the enzyme or its substrate, a colored compound, e.g., a dye, as well as other organic compounds (i.e., compounds that comprise one or more carbon atoms). In one embodiment, the sequestering agent physically separates the enzyme from its substrate by sequestering the substrate in a micelle. A “micelle” is a colloidal aggregate of amphipathic molecules which occurs at a well-defined concentration (the critical micelle concentration). A typical number of aggregated molecules in a micelle is 50 to 100. Critical micelle concentration (CMC) is the total concentration of detergent that corresponds to the maximum possible concentration of detergent monomer in solution (see FIG. 5).

A quench reagent for a particular enzyme is likely to quench enzymes in the same class. Thus, generally a quench reagent for Renilla luciferase is likely to quench other anthozoan luciferases, and a quench reagent for firefly luciferase is likely to quench other beetle luciferases. Likewise, generally, a quench reagent for an enzyme that catalyzes a particular reaction, e.g., a peroxidase or a phosphatase, is likely to quench other enzymes that catalyze that reaction, i.e., other peroxidases and other phosphatases, respectively.

Preferred substrate analog inhibitors for the compositions and kits, and for use in the methods of the invention, include, but are not limited to, substrate analog inhibitors which inhibit a luminescence reaction including those that are structurally related to the native substrate but are modified to contain a substrate for a different enzyme (a “prosubstrate”). Preferred substrate analog inhibitors include, but are not limited to, substrate analog inhibitors for anthozoan luciferases, e.g., for Renilla luciferase, including coelenterazine hh methyl ether and analogs thereof, as well as other substrate analog inhibitors for Renilla luciferase, e.g., ones that bind the enzyme but do not permit the enol oxygen to be involved in an oxidation within the active site, e.g., coelenterazine ethyl ether; peroxidases, e.g., horseradish peroxidase; and phosphatases, e.g., alkaline phosphatases, including stabilized dioxetanes that are not attached to a fluor, i.e., the analog binds enzyme but does not generate light. For instance, substrate analogs for an anthozoan luciferase include those related to a compound having the formula:

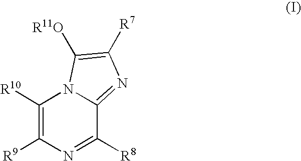

wherein R⁷ is H, alkyl, heteroalkyl, aryl, or —CH₂—C₆H₄OR¹⁴;

R⁸ is H, alkyl, heteroalkyl, or aryl;

R⁹ is H, alkyl, heteroalkyl, aryl or —C₆H₄OR¹⁵;

R¹⁰ is —H, —CH₃, or —CH(CH₃)₂;

R¹¹ is not an enzyme removable group;

R¹⁴ and R¹⁵ are independently enzyme-removable groups;

with the proviso that R¹⁴ and R¹⁵ are not all acetyl groups.

“Aryl” includes an aromatic ring, for example, an aryl or heteroaryl ring such as a phenyl or napthyl group.

In one specific embodiment, R¹¹ is C₁-C₁₀ alkylether.

In one specific embodiment, R¹¹ is methylether.

In one specific embodiment, the substrate analog is 2,8-dibenzyl-3-methoxy-6-phenyl-imidazo[1,2-a]pyrazine (coelenterazine hh methyl ether) having the formula (II):

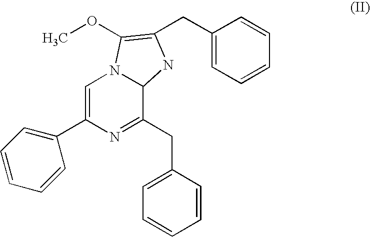

A synthesis for a compound of formula (II) includes adding, to a stirred solution of 2,8-dibenzyl-6-phenyl-7H-imidazo[1,2-a]pyrazin-3-one (0.25 g, 0.6 mmol) in dry DMF (10 mL) at ambient temperature under argon, diisopropylethylamine (1.1 mL, 6.0 mmol) all at once, followed by dropwise addition of methyl iodide (0.4 mL, 6.0 mmol). After stirring for 1 hour the reaction was complete by TLC analysis. The reaction mixture was diluted with dichloromethane (75 mL) and washed twice with water. The organic extracts were dried over anhydrous sodium sulfate, filtered and evaporated to provide a brown oil. The crude oil was purified by flash chromatography on silica gel (30 g) using dichloromethane as mobile phase. Appropriate fractions were pooled and evaporated to afford 200 mg (77%) of the desired compound.

Substrate analogs for luciferases that are modified to contain a substrate for another enzyme (a “prosubstrate”) which, in the absence of that other enzyme and the presence of the luciferase and appropriate reagents do not result in luminescence but in the presence of the other enzyme and the luciferase and appropriate reagents, yield luminescence, may be employed in the kits and methods of the invention, i.e., prosubstrates may be substrate analog inhibitors. Thus, such substrate analogs can be employed as a selective quench reagent in reactions which lack the enzyme that recognizes the prosubstrate or as a luminescent prosubstrate in reactions which contain the enzyme. For instance, those analogs include derivatives of aminoluciferin, dihydroluciferin, luciferin 6′ methyl ether, luciferin 6′ chloroethylether, or coelenterazine, e.g., derivatives of coelenterazine such as coelenterazine n, coelenterazine h, coelenterazine c, coelenterazine cp, coelenterazine e, coelenterazine f, coelenterazine fcp, coelenterazine i, coelenterazine icp or coelenterazine 2-methyl, that are modified to contain substrates for other enzymes, e.g., see PCT/US03/02936.

Generally, for coelenterazine this derivatization involves the conversion of functional groups such as phenol (—C₆H₄—OH), carbonyl (>C═O), and aniline (—C₆H₄—NH₂) into groups which are less reactive toward their surroundings. Since the normal reactivities of the functional groups are inhibited by the presence of the enzyme-removable group, the enzyme-removable group can be referred to as a protecting group. Possible protecting groups include esters, which can be removed by interaction with esterases. Possible protecting groups also include phosphoryls, which can be removed by interaction with phosphatases, including phosphodiesterases and alkaline phosphatase. Possible protecting groups also include glucosyls, which may be removed by interaction with glycosidases, α-D-galactoside, β-D-galactoside, α-D-glucoside, β-D-glucoside, α-D-mannoside, β-D-mannoside, β-D-fructofuranoside, and β-D-glucosiduronate. One skilled in the art would be able to recognize other enzyme-removable protecting groups that could be used in the invention. Examples of the interaction of enzymes and enzyme-removable groups are described in U.S. Pat. No. 5,831,102, as well as Tsien (1981); Redden et al. (1999); and Annaert et al. (1997).

Enzyme-removable groups may be designed such that they can only be removed by the action of a specific enzyme. For example, certain fatty acids may be used as enzyme-removable groups, and only specific esterases will convert these protected coelenterazines into coelenterazines. A protecting group with high steric hindrance, such as a tert-butyl group, may be used. Such a protecting group could be useful in screening for novel esterases that can act upon bulky, hindered esters. Amino acids may also be used as protecting groups. The protected coelenterazines may be further modified by substituting the enol oxygen atom with a nitrogen atom connected to a protecting group. This type of protecting group cold then be removed by a protease, and subsequent hydrolysis of the protected coelenterazine to the enol/carbonyl would provide a coelenterazine.

These enzyme-removable groups are preferably derivatives of alcohol functional groups. In the case of a carbonyl functional group in coelenterazines, derivatization may involve the conversion of the carbonyl to an enol group (—C═C—OH). The carbonyl and enol forms of the coelenterazine may be in a dynamic equilibrium in solution such that there is always a proportion of the substrates that are in the enol form. The hydroxyl (—OH) portion of the enol group can be derivatized. Derivatization via ester formation using an acylating agent is illustrated schematically below. The coelenterazine having structure III contains two phenolic groups and one carbonyl group, and any combination of these groups may be protected.

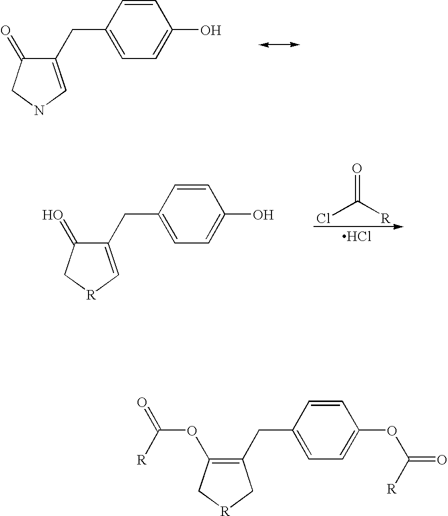

Derivatization with ether protecting groups can be carried out for example by treating the coelenterazine with an alkylating agent such as acetoxymethyl bromide. Derivatization with ester protecting groups can be carried out for example by treating the coelenterazine with an acylating agent, such as an acetic anhydride or an acetyl chloride. These derivatizations are carried out in basic conditions, that is pH between 7 and 14. Under these conditions, both the phenolic hydroxyls as well as the imidazolone oxygen can react to form the corresponding esters or ethers. The imidazolone oxygen is believed to react when in the form of the enol. Examples of the protection/deprotection process as well as various protecting groups are described in “Protective Groups in Organic Synthesis.” Eds. Greene, Wuts. John Wiley and Sons, New York, 1991.

One example of the derivatization process is the synthesis of protected coelenterazine IV from coelenterazine III. Protected coelenterazine IV is also known as triacetyl-coelenterazine due to the presence of three acetyl protecting groups.

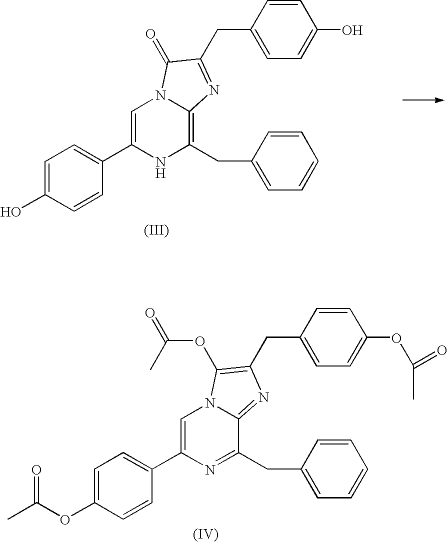

A compound having the structure of compound IV has reportedly been used as in intermediate in efforts to establish the structure of native coelenterazine III (Inoue et al., 1977). It is expected that protected coelenterazine IV would have fairly low stability relative to other protected coelenterazines, given the lability of the acetyl-derivatized enol group.

For a given protecting group, a derivatized enol is more labile than a similarly derivatized phenol. This increased ability of the enol derivative to react permits the selective hydrolysis of the enol derivative to again provide the imidazolone carbonyl. This type of compound is referred to as a partially protected species since some of the functional groups are protected while others are not. These partially protected species can be used in biological assays, or they can be further reacted with a different acylating or alkylating agent to form an unsymmetrical compound, that is a compound with more than one type of protecting group which also can be used in assays. Selection of the appropriate protecting group may depend on the cell type under consideration and on the desired rate of hydrolysis. The selective hydrolysis can be carried out, for example, as described in Inoue et al. (1977). This is illustrated in the following reaction scheme, for the selective hydroysis of triacetyl-coelenterazine (IV) to diacetyl-coelenterazine (V) and subsequent formation of an unsymmetrical protected coelenterazine (VI).

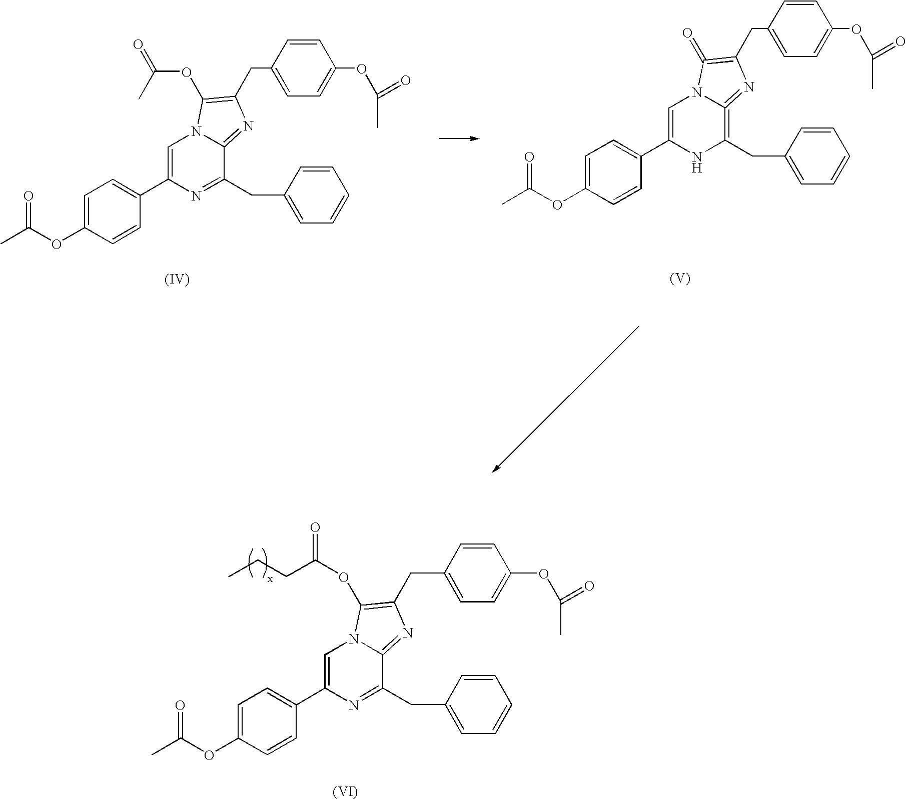

Structures VII-IX illustrate protected coelenterazines having a protecting group on the carbonyl.

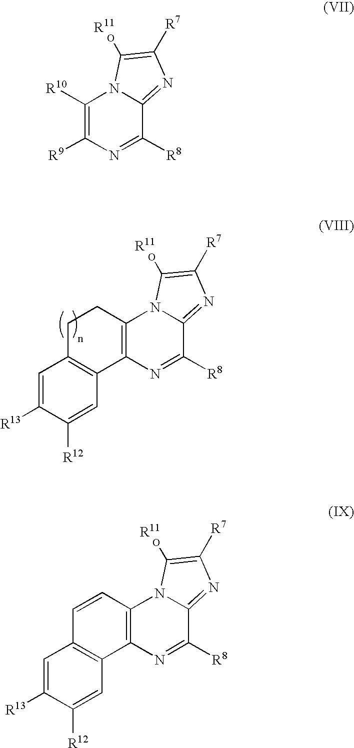

R⁷, R⁸, R⁹ and R¹⁰ can independently be H, alkyl, heteroalkyl, aryl, or combinations thereof. R¹² and R¹³ can independently be —OR¹⁶, H, OH, alkyl, heteroalkyl, aryl, or combinations thereof. For structure VIII, n can be 0, 1, or 2, preferably 1.

Preferably, R⁷ is as described for R¹ or is —CH₂—CH₄OR¹⁴.

Preferably R⁸ is as described for R², and R¹⁰ is as described for R⁴.

Preferably, R⁹ as described for R³ or is —C₆H₄OR¹⁵.

R¹¹, R¹⁴, R¹⁵, and R¹⁶, together identified as R^{P}, are protecting groups and can be independently any of a variety of protecting groups. Preferably, these species, together with their corresponding O atom, are ethers, esters, or combinations thereof. For example, the protecting group can be acetyl (R^{P}=—C(═O)—CH₃), butyryl (R^{P}=—C(═O)—C₃H₇), acetoxymethyl (R^{P}=—CH₂—O—C(═O)—CH₃), propanoyloxymethyl (R^{P}—CH₂—O—C(═O)—C₂H₅), butyryloxymethyl (R^{P}=—CH₂—O—C(═O)—C₃H₇), pivaloyloxymethyl (R^{P}=—CH₂—O—C(═O)—C(CH₃)₃), or t-butyryl (R^{P}=—C(═O)—C(CH₃)₃).

Specific examples of protected coelenterazines include triacetyl-coelenterazine (IV), tributyryl-coelenterazine (X), diacetyl-coelenterazine-h (XI), acetoxymethyl diacetyl-coelenterazine (XII), acetoxymethyl acetyl-coelenteraxine-h (XIII), pivaloyloxymethyl-coelenterazine-h (XIV), and acetoxymethyl-dideoxycoelenterazine (XV).

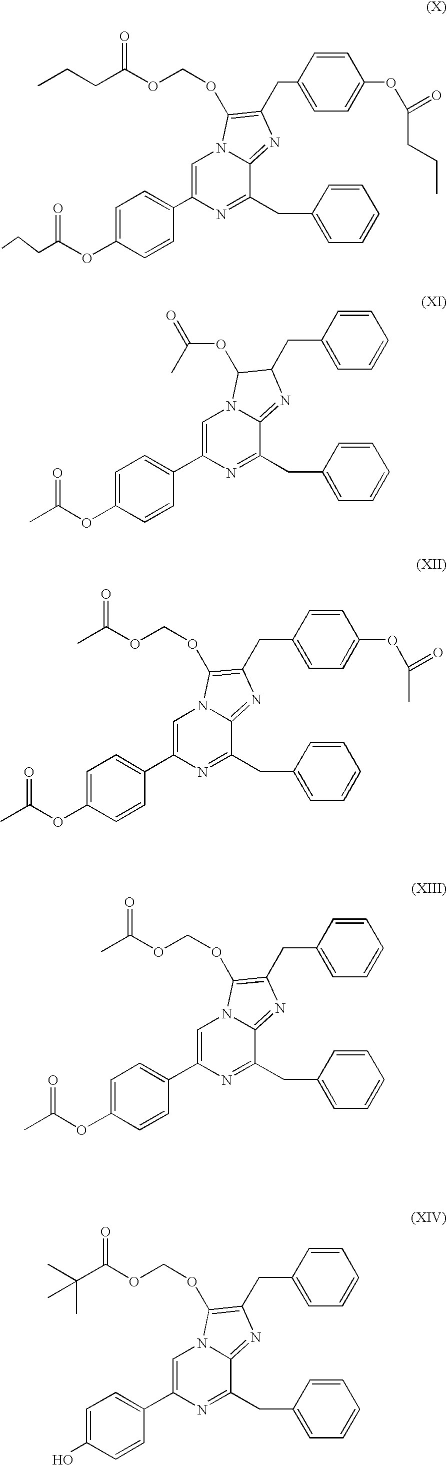

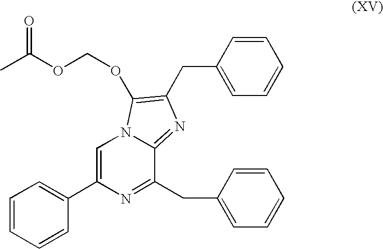

The protecting groups can be removed, and the original functional group restored, when the protected coelenterazine interacts with the appropriate deprotecting enzyme. When the appropriate deprotecting enzyme is absent, the protecting group is not removed and in some embodiments, the protected coelenterazine may be employed as an inhibitor of the luciferase. For ester and ether protecting groups, the deprotecting enzyme can for example be any hydrolase, including esterases. For coelenterazines, having the carbonyl functional group in its deprotected form (i.e., carbonyl) allows for a luminescent interaction with a luminogenic protein, including Renilla luciferase, Oplophorus luciferase, Cypridina luciferase, and aequorin. The protected coelenterazine may only need to be deprotected at the carbonyl site to be converted into a coelenterazine. The presence of protecting groups on the phenolic hydroxyls may still hinder or prevent a luminescent interaction, however.

For enzymes which employ dioxetane substrates, substrates for the reaction may include, and substrate analog inhibitors of the reaction may be structurally related to, a dioxetane-containing substrate having the formula

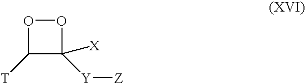

where T is a substituted (i.e., containing one or more C₁-C₇ alkyl groups or heteroatom groups, e.g. halogens) or unsubstituted cycloalkyl ring (having between 6 and 12 carbon atoms, inclusive, in the ring) or polycycloalkyl group (having 2 or more fused rings, each ring independently having between 5 and 12 carbon atoms, inclusive), bonded to the 4-membered dioxetane ring by a Spiro linkage, e.g., a chloroadamantyl or an adamantyl group, most preferably chloroadamantyl: Y is a fluorescent chromophore, (i.e. Y is group capable of absorbing energy to form an excited, i.e. higher energy, state, from which it emits light to return to its original energy state); X is hydrogen, a straight or branched chain alkyl or heteroalkyl group (having between 1 and 7 carbon atoms, inclusive, e.g., methoxy, trifluoromethoxy, hydroxyethyl, trifluoroethoxy or hydroxypropyl), an aryl group (having at least 1 ring e.g., phenyl), a heteroaryl group (having at least 1 ring e.g., pyrrolyl or pyrazolyl), a heteroalkyl group (having between 2 and 7 carbon atoms, inclusive, in the ring, e.g., dioxane), an aralkyl group (having at least 1 ring e.g., benzyl), an alkaryl group (having at least 1 ring e.g., tolyl), or an enzyme-cleavable group i.e., a group having a moiety which can be cleaved by an enzyme to yield an electron-rich group bonded to the dioxetane, e.g., phosphate, where a phosphorus-oxygen bond can be cleaved by an enzyme, e.g., acid phosphatase or alkaline phosphatase, to yield a negatively charged oxygen bonded to the dioxetane or OR; and Z is hydrogen, hydroxyl, or an enzyme-cleavable group, provided that at least one of X or Z must be an enzyme-cleavable group, so that the enzyme cleaves the enzyme-cleavable group which leads to the formation of a negatively charged group (e.g., an oxygen anion) bonded to the dioxetane, the negatively charged group causing the dioxetane to decompose to form a luminescencing substance (i.e., a substance that emits energy in the form of light) that includes group Y. The luminescent signal is detected as an indication of the activity of the enzyme. By measuring the intensity of luminescence, the activity of the enzyme can be determined.

An active substrate for a chemiluminescent reaction is generated when X, in formula XVI, is OR, moiety R is a straight or branched alkyl, aryl, cycloalkyl or arylalkyl of 1-20 carbon atoms. R may include 1 or 2 heteroatoms which may be P, N, S or O. The substituent R is halogenated. The degree of halogenation will vary depending on the selection of substituents on the adamantyl group, on the aryl group, and the desired enzyme kinetics for the particular application envisioned. Most preferably, R is a trihaloalkyl moiety. Preferred groups include trihalo lower alkyls, including trifluoroethyl, trifluoropropyl, heptafluoro butyrol, hexafluoro-2-propyl, a-trifluoromethyl benzyl, α-trifluoromethyl ethyl and difluorochloro butyl moieties. The carbon atoms of substituent R may be partially or fully substituted with halogens. When R is aryl, preferred groups may include a phenyl ring substituted with one or more chloro, fluoro, or trifluoromethyl groups, e.g., 2,5-dichlorophenyl, 2,4-difluorophenyl, 2,3,5-trifluorophenyl, 2-chloro-4-fluorophenyl or 3-trifluoromethyl phenyl. Fluorine and chlorine are particularly preferred substituents, although bromine and iodine may be employed in special circumstances.

Group Y is a fluorescent chromophore or fluorophore bonded to enzyme-cleavable group Z. Y becomes luminescent upon the dioxetane decomposition when the reporter enzyme cleaves group Z, thereby creating an electron-rich moiety which destabilizes the dioxetane, causing the dioxetane to decompose. Decomposition produces two individual carbonyl compounds, one of which contains group T, and the other of which contains groups X and Y. The energy released from dioxetane decomposition causes compounds containing the X and the Y groups to luminesce (if group X is hydrogen, an aldehyde is produced). Y preferably is phenyl or aryl. The aryl moiety bears group Z, as in formula XVI, and additionally 1-3 electron active groups, such as chlorine or methoxy, as described in U.S. Pat. No. 5,582,980.

Any chromophore can be used as Y. In general, it is desirable to use a chromophore which maximizes the quantum yield in order to increase sensitivity. Therefore, Y usually contains aromatic groups. Examples of suitable chromophores are further detailed in U.S. Pat. No. 4,978,614.

Group Z bonded to chromophore Y is an enzyme cleavable group. Upon contact with an enzyme, the enzyme-cleavable group is cleaved yielding an electron-rich moiety bonded to a chromophore Y; this moiety initiates the decomposition of the dioxetane into two individual carbonyl containing compounds e.g., into a ketone or an ester and an aldehyde if group X is hydrogen. Examples of electron-rich moieties include oxygen, sulfur, and amine or amino anions. The most preferred moiety is an oxygen anion. Examples of suitable Z groups, and the enzymes specific to these groups are given in Table 1 of U.S. Pat. No. 4,978,614. Such enzymes include alkaline and acid phosphatases, esterases, decarboxylases, phospholipase D, β-xylosidase, β-D-fucosidase, thioglucosidase, β-D-galactosidase, α-D-galactosidase, α-D-glucosidase, β-D-glucosidase. β-D-glucouronidase α-D-mannosidase, β-D-mannosidase, β-D-fructofuranosidase, β-D-glucosiduronase, and trypsin.

Dioxetane analogs may also contain one or more solubilizing substituents attached to any of the T, Y and X, i.e., substituents which enhance the solubility of the analogs in aqueous solution. Examples of solubilizing substituents include carboxylic acids, e.g., acetic acid; sulfonic acids, e.g., methanesulfonic acid; and quaternary amino salts, e.g., ammonium bromide; the most preferred solubilizing substituent is methane or ethanesulfonic acid. Other dioxetanes from which dioxetane analogs useful in the practice of this invention may be prepared are described in U.S. Pat. No. 5,089,630; U.S. Pat. No. 5,112,960; U.S. Pat. No. 5,538,847 and U.S. Pat. No. 5,582,980.

In one embodiment, the substrate analog for a first enzyme-mediated luminescence reaction is not a substrate analog for a beetle luciferase, e.g., the substrate analog is not dehydroluciferin, ATP, benzothiazole, 1-(4-amino phenyl)-6-methylbenzothiazole, 2-phenyl benzothiazole, or 2(O-hydroxyphenyl)benzothiazole.

In one embodiment, preferred substrate analogs are irreversible competitive inhibitors of the native substrate.

Preferred sequestering agents include surfactants and detergents, e.g., those which, in an effective amount, physically separate a substrate from its corresponding enzyme so that, preferably, no enzymatic reaction can occur, as well as antibodies or other ligands for the substrate or the enzyme. Preferred sequestering agents include agents which sequester at least a portion, e.g., 35% or more, for instance 50%, 60%, 70%, 80%, 90% or more, of the substrate for a first enzyme, but not a second, distinct enzyme and its corresponding substrate, e.g., into micelles, or shifts the solubility of the first substrate or first enzyme but not that of a second, distinct substrate and its corresponding enzyme, so as to inhibit, e.g., inhibit by at least 35% or more, for instance 50%, 60%, 70%, 80%, 90% or more, an interaction between the first substrate and first enzyme which results in light generation. Preferred sequestering agents, include, but are not limited to, anionic, nonionic, amphiteric or cationic detergents or surfactants including those in FIG. 5. In one embodiment, preferred sequestering agents include, but are not limited to, crown ethers, ethoxylated Tomahs such as Tomah E®, azacrown ether, cyclodextran, Tween® 20 (poly(oxyethylene)ₓ-sorbitane-monolaurate), Tween 80, Big Chaps, CHAPS, DTAB, Triton® X-100 (alkylpolyether alcohol; [C₁₆H₂₆O₂]ₙ), and Tergitol®, e.g., Tergitol® NP-9, polyvinylpyrolidone, and glycols, e.g., polyethylene glycol, e.g., 400 or 600. Thus, for instance, the addition of an agent that physically separates a substrate, e.g., a majority of a substrate, from a corresponding enzyme may sequester the substrate (e.g., coelenterazine) in micelles while the enzyme, e.g., Renilla luciferase, remains in the aqueous portion of the solution. In particular, a preferred sequestering agent for a first luminescent reaction is one which physically separates at least a majority of a substrate for a first enzyme which mediates a luminescence reaction from the enzyme, and does not substantially quench the light from a second, distinct enzyme-mediated luminescent reaction. In one embodiment, the sequestering agent for a first anthozoan luciferase-mediated reaction may be a charged detergent, e.g., about 0.05%, 0.1%, 1.0%, 2% v/v or greater CHAPS, for instance, when a second enzyme-mediated luminescence reaction is mediated by a firefly luciferase such as Ppe2 (WO 01/20002). In another embodiment, the sequestering agent for a first anthozoan luciferase-mediated reaction may be Triton X-100 or Tergitol® NP-9, e.g., 0.01%, 0.05%, 0.1%, 0.5%, 1.0%, 2% and greater Triton X-100 or Tergitol® NP-9, for instance, when a second enzyme-mediated luminescence reaction is mediated by Luc+ (U.S. Pat. No. 5,670,356).

In one embodiment, preferred colored compounds are those which quench blue, green or red light. Compounds may be screened by eye or by absorption spectra to identify candidates for compounds which quench blue, green or red light (see, The Sigma-Aldrich Handbook of Stains, Dyes and Indicaters, Green, ed., Aldrich Chemical Company, Milwaukee, Wis., 1990, which is specifically incorporated by reference herein).

Red light as used herein includes light of wavelengths longer than about 590 nm and less than about 730 nm, e.g., wavelengths of 610 nm to 650 nm. Yellow-green light as used herein includes light of wavelengths from about 490 nm to about 590 nm, preferably from about 520 nm to about 570 nm. Yellow light as used herein includes light of wavelengths greater than 560 nm to about 590 nm. Green light as used herein includes light of wavelengths greater than 490 nm to about 560 nm. Blue light as used herein includes light of wavelengths greater than 400 nm to about 490 nm.

For example, red light may correspond to a wavelength of 700 nm, a frequency of 4.29×10¹⁴ Hz or 1.77 eV, as well as to a wavelength of 650 nm, a frequency of 4.62×10¹⁴ Hz or 1.91 eV. Yellow light may correspond to a wavelength of 580 nm, a frequency of 5.16×10¹⁴ Hz or an energy of 2.14 eV. Green light may correspond to a wavelength of 550 nm, a frequency of 5.45×10¹⁴ Hz or an energy of 2.25 eV. Blue light may correspond to a wavelength of 450 nm, a frequency of 6.66×10¹⁴ Hz or an energy of 2.75 eV, while purple light may correspond to a wavelength of 400 nm, a frequency of 7.50×10¹⁴ Hz or an energy of 3.10 eV.

For instance, yellow compounds are useful to quench blue light such as the light emitted by Renilla luciferase- and horseradish peroxidase-mediated reactions. Moreover, yellow compounds do not quench the green-yellow light emitted by some beetle luciferases and so they may be used to quench a dual assay such as a Renilla luciferase/firefly luciferase assay. Preferred yellow compounds include, but are not limited to, those which, when dissolved in an aqueous solution, have a peak absorbance within 75 nm of 560 to 590 run, such as dipyridamole and berberine hemisulfate. Preferred compounds to quench light emitted by Renilla luciferase include, but are not limited to, compounds that absorb blue light and, in one embodiment, permit yellow-green light to be transmitted, including but not limited to acridine orange, basic orange 21, 4-(4-dimethylaminophenylazo)benzenenearsonic acid hydrochloride, 5-aminofluorescein, bis[N,N-bis(carboxymethyl)aminomethyl]fluoresceine, 2,4-diamino-5-(2-hydroxy-5-nitrophenylazo)benzenesulfonic acid, Nubian yellow TB, acid orange 10, rosolic acid, and solvent yellow 14.

In another embodiment, preferred compounds include compounds which quench red light, e.g., those compounds which, in solution, are cyan or blue colored, including but not limited to azure B tetrafluoroborate, acid blue 93, 5,5′,7-indigotrisulfinic acid tripotassium salt, cresyl violet acetate, tryptan blue, Twort stain, and lissamine green B. Preferred blue compounds are those which, when dissolved in an aqueous solution, have a peak absorbance within 75 nm of 400 to 490 nm.

Blue compounds for quenching red or yellow, but not blue, light include, but are not limited to, blue chromate dye, isosulfan blue, methylene blue, Coomassie blue, acid blue, orcein, Prussian blue, potassium indigotrisulfonate, alpha-napthophthalein, azure II, oil blue N, patent blue VF, pararoaniline base, rhodanile blue, tetrabromophenol blue, toluidine blue O, Victoria pure blue BO, Victoria Blue B, alkali blue 6B, alphazurine A, and cyanine dye.

In yet another embodiment, preferred compounds include compounds which quench green light, e.g., those compounds which in solution are magenta or red colored, and, in one embodiment permit red light to be transmitted, including but not limited to, acid blue, acid violet 19, amido naphthol red 6B, and basic red 9. In one preferred embodiment, compounds which quench green light and transmit blue light include acid violet 17, indigo blue, pinacyanol chloride, rhodamine 6G perchlorate, rhodanile blue, pararosanaline base, rose Bengal bis(triethylammonium) salt, and 3,3′-dimethylphenolphthalein. Preferred compounds are those which, when dissolved in an aqueous solution, have a peak absorbance within 75 nm of 590 to 730 nm.

In one embodiment, suitable compounds useful as a quench reagent for chemiluminescent reactions include organic compounds (i.e. compounds that comprise one or more carbon atoms), such as those disclosed in U.S. application Ser. No. 09/590,884, the disclosure of which is incorporated by reference herein. Suitable organic compounds can comprise a carbon-sulfur bond or a carbon-selenium bond, for example suitable organic compounds can comprise a carbon-sulfur double bond (C═S), a carbon selenium double bond (C═Se), a carbon-sulfur single bond (C—S), or carbon-selenium single bond (C—Se). Suitable organic compounds can also comprise a carbon bound mercapto group (C—SH) or a sulfur atom bound to two carbon atoms (C—S—C). Preferred compounds are lipophyllic in nature.

Suitable compounds that comprise a carbon sulfur double bond or a carbon selenium double bond include for example compounds of formula (XVII):

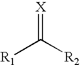

wherein X is S or Se; R₁ and R₂ are each independently hydrogen, (C₁-C₂₀)alkyl, (C₃-C₈)cycloalkyl, (C₁-C₂₀)alkoxy, (C₂-C₂₀)alkenyl, (C₂-C₂₀)alkynyl, aryl, heteroaryl, or NRₐR_{b}; or R₁ and R₂ together with the carbon to which they are attached form a 5, 6, 7, or 8 membered saturated or unsaturated ring comprising carbon and optionally comprising 1, 2, or 3 heteroatoms selected from oxy (—O—), thio (—S—), or nitrogen (—NR_{c})—, wherein said ring is optionally substituted with 1, 2, or 3 halo, hydroxy, oxo, thioxo, carboxy, (C₁-C₂₀)alkyl, (C₃-C₈)cycloalkyl, (C₁-C₂₀)alkoxy, (C₁-C₂₀)alkanoyl, (C₁-C₂₀)alkoxycarbonyl, (C₂-C₂₀)alkenyl, (C₂-C₂₀)alkynyl, aryl, or heteroaryl; and Rₐ, R_{b} and R_{c} are each independently hydrogen, (C₁-C₂₀)alkyl, (C₃-C₈)cycloalkyl, (C₂-C₂₀)alkenyl, (C₁-C₂₀)alkanoyl, (C₁-C₂₀)alkoxycarbonyl, (C₂-C₂₀)alkynyl, aryl, heteroaryl; wherein any (C₁-C₂₀)alkyl, (C₃-C₈)cycloalkyl, (C₁-C₂₀)alkoxy, (C₂-C₂₀)alkenyl (C₁-C₂₀)alkanoyl, (C₁-C₂₀)alkoxycarbonyl, or (C₂-C₂₀)alkynyl of R₁, R₂, Rₐ, R_{b}, and R_{c} is optionally substituted with one or more (e.g., 1, 2, 3, or 4) halo, hydroxy, mercapto, oxo, thioxo, carboxy, (C₁-C₂₀)alkanoyl, (C₁-C₂₀)alkoxycarbonyl, aryl, or heteroaryl; and wherein any aryl or heteroaryl is optionally substituted with one or more (1, 2, 3, or 4) halo, hydroxy, mercapto, carboxy, cyano, nitro, trifluoromethyl, trifluoromethoxy, (C₁-C₂₀)alkanoyl, (C₁-C₂₀)alkanoyloxy, sulfo or (C₁-C₂₀)alkoxycarbonyl; or a salt thereof.

The term “halo” as used herein denotes fluoro, chloro, bromo, or iodo.

The terms “Alkyl”, “alkoxy”, “alkenyl”, “alkynyl”, etc. as used herein denote both branched and unbranched groups; but reference to an individual radical such as “propyl” embraces only the straight, unbranched chain radical, a branched chain isomer such as “isopropyl” being specifically referred to.

The term “Aryl”, as used herein, denotes a monocyclic or polycyclic hydrocarbon radical comprising 6 to 30 atoms wherein at least one ring is aromatic. Preferably, aryl denotes a phenyl radical or an ortho-fused bicyclic carbocyclic radical having about nine to ten ring atoms in which at least one ring is aromatic. “Heteroaryl” encompasses a radical of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of a polycyclic ring comprising 8 to 30 atoms derived therefrom. Preferably, heteroaryl encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

Suitable compounds that comprise a mercapto group include for example compounds of the formula R₃SH wherein: R₃ is (C₁-C₂₀)alkyl, (C₃-C₈)cycloalkyl, (C₂-C₂₀)alkenyl, (C₂-C₂₀)alkynyl, aryl, or heteroaryl; wherein any (C₁-C₂₀)alkyl, (C₃-C₈)cycloalkyl, (C₂-C₂₀)alkenyl, or (C₂-C₂₀)alkynyl of R₃ is optionally substituted with one or more (e.g 1, 2, 3, or 4) halo, hydroxy, mercapto oxo, thioxo, carboxy, (C₁-C₂₀)alkanoyl, (C₁-C₂₀)alkoxycarbonyl, aryl, heteroaryl, or NR_{d}Rₑ; wherein R_{d} and Rₑ are each independently hydrogen, (C₁-C₂₀)alkyl, (C₃-C₈)cycloalkyl, (C₂-C₂₀)alkenyl, (C₂-C₂₀)alkynyl, (C₁-C₂₀)alkanoyl, (C₁-C₂₀)alkoxycarbonyl aryl, or heteroaryl; and wherein any aryl or heteroaryl is optionally substituted with one or more (1, 2, 3, or 4) halo, mercapto, hydroxy, oxo, carboxy, cyano, nitro, trifluoromethyl, trifluoromethoxy, (C₁-C₂₀)alkanoyl, (C₁-C₂₀)alkanoyloxy, sulfo or (C₁-C₂₀)alkoxycarbonyl; or a salt thereof.

Other suitable compounds include for example compounds of the formula R₄NCS wherein: R₄ is (C₁-C₂₀)alkyl, (C₃-C₈)cycloalkyl, (C₂-C₂₀)alkenyl, (C₂-C₂₀)alkynyl, aryl, or heteroaryl; wherein any (C₁-C₂₀)alkyl, (C₃-C₈)cycloalkyl, (C₂-C₂₀)alkenyl, or (C₂-C₂₀)alkynyl of R₃ is optionally substituted with one or more (e.g 1, 2, 3, or 4) halo, hydroxy, mercapto oxo, thioxo, carboxy, (C₁-C₂₀)alkanoyl, (C₁-C₂₀)alkoxycarbonyl, aryl, heteroaryl, or NR_{f}R_{g}; wherein R_{f} and R_{g} are each independently hydrogen, (C₁-C₂₀)alkyl, (C₃-C₈)cycloalkyl, (C₂-C₂₀)alkenyl, (C₂-C₂₀)alkynyl, (C₁-C₂₀)alkanoyl, (C₁-C₂₀)alkoxycarbonyl aryl, or heteroaryl; and wherein any aryl or heteroaryl is optionally substituted with one or more (1, 2, 3, or 4) halo, mercapto, hydroxy, oxo, carboxy, cyano, nitro, trifluoromethyl, trifluoromethoxy, (C₁-C₂₀)alkanoyl, (C₁-C₂₀)alkanoyloxy, sulfo or (C₁-C₂₀)alkoxycarbonyl; or a salt thereof.

Other suitable compounds that comprise a carbon-selenium single bond or a carbon sulfur single bond include compounds of formula R₅—X—R₆ wherein:

X is —S— or —Se—;

R₅ is (C₁-C₂₀)alkyl, (C₃-C₈)cycloalkyl, (C₂-C₂₀)alkenyl, (C₂-C₂₀)alkynyl, aryl, or heteroaryl; and R₆ is hydrogen, (C₁-C₂₀)alkyl, (C₃-C₈)cycloalkyl, (C₂-C₂₀)alkenyl, (C₂-C₂₀)alkynyl, aryl, or heteroaryl;

or R₅ and R₆ together with X form a heteroaryl;

wherein any (C₁-C₂₀)alkyl, (C₃-C₈)cycloalkyl, (C₂-C₂₀)alkenyl, or (C₂-C₂₀)alkynyl of R₅ or R₆ is optionally substituted with one or more (e.g 1, 2, 3, or 4) halo, hydroxy, mercapto oxo, thioxo, carboxy, (C₁-C₂₀)alkanoyl, (C₁-C₂₀)alkoxycarbonyl, aryl, heteroaryl, or NRₖRₘ;

wherein Rₖ and Rₘ are each independently hydrogen, (C₁-C₂₀)alkyl, (C₃-C₈)cycloalkyl, (C₂-C₂₀)alkenyl, (C₂-C₂₀)alkynyl, (C₁-C₂₀)alkanoyl, (C₁-C₂₀)alkoxycarbonyl aryl, or heteroaryl; and

wherein any aryl or heteroaryl is optionally substituted with one or more (1, 2, 3, or 4) halo, mercapto, hydroxy, oxo, carboxy, cyano, nitro, trifluoromethyl, trifluoromethoxy, (C₁-C₂₀)alkanoyl, (C₁-C₂₀)alkanoyloxy, sulfo or (C₁-C₂₀)alkoxycarbonyl; or a salt thereof.

Specific and preferred values listed below for radicals, substituents, and ranges, are for illustration only; they do not exclude other defined values or other values within defined ranges for the radicals and substituents Specifically, (C₁-C₂₀)alkyl can be methyl, ethyl, propyl, isopropyl, butyl, iso-butyl, sec-butyl, pentyl, 3-pentyl, or hexyl; (C₃-C₈)cycloalkyl can be cyclopropyl, cyclobutyl, cyclopentyl, or cyclohexyl; (C₁-C₂₀)alkoxy can be methoxy, ethoxy, propoxy, isopropoxy, butoxy, iso-butoxy, sec-butoxy, pentoxy, 3-pentoxy, or hexyloxy; (C₂-C₂₀)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₂₀)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₂₀)alkanoyl can be acetyl, propanoyl or butanoyl; (C₁-C₂₀)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₂-C₂₀)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

Specifically, R₁ and R₂ can each independently be hydrogen, (C₁-C₂₀)alkyl, (C₃-C₈)cycloalkyl, (C₂-C₂₀)alkenyl, (C₂-C₂₀)alkynyl, aryl, heteroaryl, or NRₐR_{b}; wherein Rₐ and R_{b} are each independently hydrogen, (C₁-C₂₀)alkyl, (C₃-C₈)cycloalkyl, (C₂-C₂₀)alkenyl, (C₁-C₂₀)alkanoyl, (C₁-C₂₀)alkoxycarbonyl, (C₂-C₂₀)alkynyl, aryl, or heteroaryl; wherein any (C₁-C₂₀)alkyl, (C₃-C₈)cycloalkyl, (C₁-C₂₀)alkoxy, (C₂-C₂₀)alkenyl (C₁-C₂₀)alkanoyl, (C₁-C₂₀)alkoxycarbonyl, or (C₂-C₂₀)alkynyl of R₁, R₂, Rₐ, and R_{b} is optionally substituted with 1 or 2 halo, hydroxy, mercapto, oxo, thioxo, carboxy, (C₁-C₂₀)alkanoyl, (C₁-C₂₀)alkoxycarbonyl, aryl, or heteroaryl; and wherein any aryl or heteroaryl is optionally substituted with one or more halo, hydroxy, mercapto, carboxy, cyano, nitro, trifluoromethyl, trifluoromethoxy, (C₁-C₂₀)alkanoyl, (C₁-C₂₀)alkanoyloxy, sulfo or (C₁-C₂₀)alkoxycarbonyl.

Specifically, R₁ and R₂ can each independently be hydrogen, (C₁-C₁₀)alkyl, (C₂-C₁₀)alkenyl, (C₂-C₁₀)alkynyl, aryl, or NRₐR_{b}.

Specifically, R₁ and R₂ together with the carbon to which they are attached can form a 5 or 6 membered saturated or unsaturated ring comprising carbon and optionally comprising 1 or 2 heteroatoms selected from oxy (—O—), thio (—S—), or nitrogen (—NR_{c})—, wherein said ring is optionally substituted with 1, 2, or 3 halo, hydroxy, oxo, thioxo, carboxy, (C₁-C₂₀)alkyl, (C₃-C₈)cycloalkyl, (C₁-C₂₀)alkoxy, (C₁-C₂₀)alkanoyl, (C₁-C₂₀)alkoxycarbonyl, (C₂-C₂₀)alkenyl, (C₂-C₂₀)alkynyl, aryl, or heteroaryl; wherein R_{c} is hydrogen, (C₁-C₂₀)alkyl, (C₃-C₈)cycloalkyl, (C₂-C₂₀)alkenyl, (C₁-C₂₀)alkanoyl, (C₁-C₂₀)alkoxycarbonyl, (C₂-C₂₀)alkynyl, aryl, heteroaryl; wherein any (C₁-C₂₀)alkyl, (C₃-C₂₀)cycloalkyl, (C₁-C₂₀)alkoxy, (C₂-C₂₀)alkenyl (C₁-C₂₀)alkanoyl, (C₁-C₂₀)alkoxycarbonyl, or (C₂-C₂₀)alkynyl of R₁, R₂, and R_{c} is optionally substituted with one or more halo, hydroxy, mercapto, oxo, thioxo, carboxy, (C₁-C₂₀)alkanoyl, (C₁-C₂₀)alkoxycarbonyl, aryl, or heteroaryl; and wherein any aryl or heteroaryl is optionally substituted with one or more halo, hydroxy, mercapto, carboxy, cyano, nitro, trifluoromethyl, trifluoromethoxy, (C₁-C₂₀)alkanoyl, (C₁-C₂₀)alkanoyloxy, sulfo or (C₁-C₂₀)alkoxycarbonyl.

Specifically, R₁ and R₂ can each independently be NRₐR_{b}; wherein Rₐ and R_{b} are each independently hydrogen, (C₁-C₂₀)alkyl, (C₃-C₈)cycloalkyl, (C₂-C₂₀)alkenyl, (C₁-C₂₀)alkanoyl, (C₁-C₂₀)alkoxycarbonyl, (C₂-C₂₀)alkynyl, aryl, heteroaryl; wherein any (C₁-C₂₀)alkyl, (C₃-C₈)cycloalkyl, (C₂-C₂₀)alkenyl (C₁-C₂₀)alkanoyl, (C₁-C₂₀)alkoxycarbonyl, or (C₂-C₂₀)alkynyl is optionally substituted with one or more halo, hydroxy, mercapto, oxo, thioxo, carboxy, aryl, or heteroaryl; and wherein any aryl or heteroaryl is optionally substituted with one or more halo, hydroxy, mercapto, carboxy, cyano, nitro, trifluoromethyl, trifluoromethoxy, (C₁-C₂₀)alkanoyl, (C₁-C₂₀)alkanoyloxy, sulfo or (C₁-C₂₀)alkoxycarbonyl.

Specifically, R₁ and R₂ can each independently be amino, (C₁-C₂₀)alkyl, (C₁-C₂₀)alkylamino, allylamino, 2-hydroxyethylamino, phenylamino, or 4-thiazoylamino.

Specifically, R₁ and R₂ can each independently be amino, methyl, allylamino, 2-hydroxyethylamino, phenylamino, or 4-thiazoylamino.

A specific value for R₃ is (C₁-C₂₀)alkyl optionally substituted with one or more halo, mercapto oxo, thioxo, carboxy, (C₁-C₂₀)alkanoyl, (C₁-C₂₀)alkoxycarbonyl, aryl, heteroaryl, or NR_{d}Rₑ.

A specific value for R₃ is 2-aminoethyl, 2-amino-2-carboxyethyl, or 2-acylamino-2-carboxyethyl.

A specific value for R₄ is aryl, optionally substituted with one or more halo, mercapto, hydroxy, oxo, carboxy, cyano, nitro, trifluoromethyl, trifluoromethoxy, (C₁-C₂₀)alkanoyl, (C₁-C₂₀)alkanoyloxy, sulfo or (C₁-C₂₀)alkoxycarbonyl.

Specifically, R₅ is (C₁-C₁₀)alkyl, (C₃-C₆)cycloalkyl, (C₂-C₁₀)alkenyl, (C₂-C₁₀)alkynyl, aryl, or heteroaryl; and R₆ is hydrogen, (C₁-C₁₀)alkyl, (C₃-C₆)cycloalkyl, (C₂-C₁₀)alkenyl, (C₂-C₁₀)alkynyl, aryl, or heteroaryl.

Specifically, R₅ and R₆ together with X form a heteroaryl.

Preferred organic compounds exclude polypeptides and proteins comprising one or more mercapto (C—SH) groups.

Preferred organic compounds exclude compounds that comprise one or more mercapto (C—SH) groups.

In one embodiment, preferably the quench reagent is not iodide, iodine, sulfate, nitrate, iso-propanol, 2-(4-aminophenyl)-6-methylbenzothiazole (APBNH), dimethyldecylphosphine oxide, pyrophosphate, benzothiazole, 2-phenylbenzothiazole, n-butanol, trans-1,2,-diaminocyclohexane-N,N,N′,N′-tetraacetic acid (CDTA), 2-(6′-hydroxy-2′-benzothiazolyl)-thiazole-4-carboxylic acid, ethylenediaminetetrethylenediaminetetraacetic acid, 2(o-hydroxyphenyl)benzothiazole, adenosine 5′-triphosphate, 2′,3′-acyclic dialcohol periodate oxidized borohydride reduced, sodium dodecyl sulfate (SDS), citric acid, Tween® 20, or Triton® X-100. In another embodiment, the composition comprising the quench reagent does not comprise citric acid, n-butanol, isopropanol, ethanol, iodide, iodine, Tween® 20, Triton® X-100, cetyl trimethyl ammonium bromide, or any combination thereof. In another embodiment, the quench reagent is not a thiol. In yet another embodiment, the quench reagent is not a selective quench reagent for a beetle luciferase.

The invention also includes single reporter and dual reporter assay kits which contain one or more selective quench reagents. The single reporter kit comprises at least one selective quench reagent composition capable of quenching photon emission from an enzyme-mediated luminescence reaction. The at least one selective quench reagent composition is disposed within a suitable first container. At least one functional enzyme substrate for the enzyme-mediated luminescence reaction is optionally included in the kit, along with a suitable second container into which the at least one functional enzyme substrate is disposed. The kit also includes instructions on its use.

In one embodiment, two or more selective quench reagents are employed in the methods, compositions and kits of the invention and, preferably, their combined effect on quenching is more than additive.

The dual reporter kit includes at least one selective quench reagent capable of quenching photon emission from at least one enzyme-mediated luminescence reaction but not capable of substantially quenching at least one second and distinct enzyme-mediated luminescence reaction. Alternatively, or in addition to at least one selective quench reagent, the kit includes a quench-and-activate composition comprising at least one first quench reagent capable of selectively quenching photon emission from at least one enzyme-mediated luminescence reaction but not capable of substantially quenching photon emission from a second and distinct enzyme-mediated luminescence reaction. The at least one selective quench reagent composition, or the quench-and-activate composition, is disposed within a suitable first container. At least one functional enzyme substrate for the first enzyme-mediated luminescence reaction is contained within a suitable second container. Optionally, the dual reporter kit comprises at least one functional enzyme substrate for the second enzyme-mediated luminescence reaction contained within a suitable third container. The dual reporter kit also includes instructions for its use. Also optionally, the dual reporter kit may also contain at least a second quench reagent, which is different than the first selective quench reagent, contained within a suitable third container. The second quench reagent, which may be a selective quench reagent, is capable of quenching the second and distinct enzyme-mediated luminescent reaction.

The invention also includes assay kits for carrying out the methods of the invention. Such kits comprise, in one or more containers, usually conveniently packaged to facilitate use in assays, quantities of various compositions for carrying out the methods. Thus, in kits for assaying for beetle luciferase, a luciferase substrate or ATP, there will be a composition that may contain one or more or any combination of the following: magnesium ion, ATP, beetle luciferase, luciferin, and/or a thiol reagent. In one embodiment, such composition may comprise both CoA and a thiol reagent, such as dithiothreitol (DTT), other than CoA, and may comprise other components, such as, for example, a proteinaceous luciferase-activity enhancer (e.g., bovine serum albumin or glycol in purified enzyme preparations), EDTA or CDTA, a phosphate salt or 2-aminoethanol, or a buffer to provide a solution at a pH and ionic strength at which the beetle luciferase-luciferin reaction will proceed at a suitable rate.

One component of such kits and compositions may be a cation, e.g., magnesium, calcium, manganese and the like.

The thiol reagents used in the methods and compositions of the invention are CoA or thiol reagents other than CoA. The thiol reagents other than CoA are reagents which have a free sulfhydryl group that is capable of being effective as a reducing agent in an air-saturated aqueous solution under conditions, of temperature, pH, ionic strength, chemical composition, and the like, at which the reaction occurs. Preferred among these reagents is DTT. Among others which can be employed are beta-mercaptoethanol, 2-mercaptopropanol (either enantiomer or both enantiomers in any combination), 3-mercaptopropanol, 2,3-dithiopropanol, and glutathione.

In kits assaying for an anthozoan luciferase, e.g., a Renilla luciferase, reaction, the composition comprises a reagent buffer, e.g., at pH 5, high salt, e.g., about 0.5 M KCl or NaCl, a substrate such as coelenterazine or coelenterazine hh, and may comprise other components.

The assay kits may also comprise one or more substrates, e.g., a substrate for the first reaction and a substrate for the second reaction, e.g., a substrate for an enzyme that yields a product which is a substrate for a luminescence reaction. The substrate may be prepared synthetically. For instance, modified forms of coelenterazine or other luciferins, “protected” forms, as described herein may be employed in the kits and methods of the invention. Protected luciferins such as protected coelenterazine include modified forms of luciferin that no longer interact with a luciferase to yield luminescence. In one embodiment, the modification is the addition of any enzyme-removable group to the luciferin and the interaction of the protected luciferin with an appropriate enzyme yields an active luciferin capable of luminescence. The enzyme which converts the protected luciferin into an active luciferin is preferably a non-luminogenic enzyme. All of the coelenterazines disclosed in WO 03/040100, the disclosure of which is incorporated by reference herein, may be converted into protected coelenterazines.

The various components described above can be combined, e.g., in solution or a lyophilized mixture, in a single container or in various combinations (including individually) in a plurality of containers. In a preferred kit for assaying for an enzyme, substrate or cofactor via an enzyme-mediated luminescence reaction in cells in which the enzyme, cofactor or substrate may be present, a solution (or the components for preparing a solution) useful for lysing the cells while preserving (against the action of various enzymes released during lysis) the enzyme, substrate or cofactor that might be in the cells in an active form, or a form which can be made active, is included.

The skilled are also aware that compositions including those described herein, and other than those described herein, may be present in any assay reaction mixture, and thus in the kits of the invention, in order to, for example, maintain or enhance the activity of an enzyme or as a consequence of the procedures used to obtain the aliquot of sample being subjected to the assay procedures. Thus, typically buffering agents, such as tricine, HEPPS, HEPES, MOPS, Tris, glycylglycine, a phosphate salt, or the like, will be present to maintain pH and ionic strength; a proteinaceous material, such as a mammalian serum albumin (preferably bovine serum albumin) or lactalbumin or an ovalbumin, that enhances the activity of an enzyme, may be present; EDTA or CDTA (cyclohexylenediaminetetraacetate) or the like, may be present, to suppress the activity of metal-containing proteases or phosphatases that might be present in systems (e.g., cells) from which the reporter to be assayed is extracted and that could adversely affect the reporter or other components of the reaction. Glycerol or ethylene glycol, which stabilize enzymes, might be present.

For instance, counterions to a cation, e.g., magnesium, may be present. As the skilled will understand, the chemical identities and concentrations of these counterions can vary widely, depending on the magnesium salt used to provide the magnesium ion, the buffer employed, the pH of the solution, the substance (acid or base) used to adjust the pH, and the anions present in the solution from sources other than the magnesium salt, buffer, and acid or base used to adjust pH. In one embodiment, the magnesium ion can be supplied as the carbonate salt, to provide the desired magnesium ion concentration, in a solution with the buffer to be used (e.g., tricine) and then the pH of the buffered solution can be adjusted by addition of a strong acid, such as sulfuric, which will result in loss of most of the carbonate (and bicarbonate) as carbon dioxide and replacement of these anions with sulfate, bisulfate, tricine anion, and possibly also other types of anions (depending on other substances (e.g., phosphate salts) that provide anions and might be present in the solution). Oxygen-saturation from the air of the solution in which the assay method is carried out is sufficient to provide the molecular oxygen required in the luciferase reaction. In any case, it is well within the skill of the ordinarily skilled to readily ascertain the concentrations of the various components in an assay reaction mixture, including the components specifically recited above in the description of the method, that are effective for activity of the luciferase.

The test kits of the invention can also include, as well known to the skilled, various controls and standards, such as solutions of known enzyme, substrate or cofactor, e.g., ATP, concentration, including no enzyme, no substrate or no cofactor (e.g., no ATP which is for a firefly luciferase negative control) solutions, to ensure the reliability and accuracy of the assays carried out using the kits, and to permit quantitative analyses of samples for the analytes (e.g., enzyme, substrate, cofactor and the like) of the kits.

The types of samples which can be assayed in accordance with the method of the invention include, among others, samples which include a luminescent reporter as a genetic reporter, a luminescent reporter as a reporter for a cellular molecule or a modulator of that molecule, a reporter in an immunoassay or a reporter in a nucleic acid probe hybridization assay. As understood in the immunoassay and nucleic acid probe arts, the enzyme assayed in accordance with the present invention is physically, e.g., chemically or recombinantly, linked, by any of numerous methods known in those arts, to an antibody or fragment thereof or nucleic acid probe used in detecting an analyte in an immunoassay or nucleic acid probe hybridization assay, respectively. Then, also following well known methods, the reporter-labeled antibody or nucleic acid probe is combined with a sample to be analyzed, to become bound to a molecule (e.g., antigen or an anti-antigen antibody, in the case of an immunoassay, or a target nucleic acid, in the case of a nucleic acid probe hybridization assay) that is sought to be detected and might be present in the sample and then reporter-labeled antibody or nucleic acid probe that did not become bound to analyte is separated from that, if any, which did become bound. The reporter can remain physically linked to the labeled antibody or probe during the assay for the reporter in accordance with the present invention or, again by known methods, can be separated from the antibody or nucleic acid probe prior to the assay for the reporter in accordance with the present invention. Immunoassays and nucleic acid probe hybridization assays, in which an enzyme that mediates a luminescence reaction can be used as a reporter or label, have many practical and research uses in biology, biotechnology, and medicine, including detection of pathogens, detection of genetic defects, diagnosis of diseases, and the like.

Another type of sample which can be assayed for the presence of a reporter in accordance with the method of the invention is an extract of cells in which expression of the reporter occurs in response to activation of transcription from a promoter, or other transcription-regulating element, linked to a DNA segment which encodes the reporter, or as a result of translation of RNA encoding the reporter. In such cells, luminescent reporters are used, similarly to the way other enzymes, such as chloramphenicol acetyltransferase, have been used to monitor genetic events such as transcription or regulation of transcription. Such uses of luminescent reporters are of value in molecular biology and biomedicine and can be employed, for example, in screening of compounds for therapeutic activity by virtue of transcription-activating or transcription-repressing activity at particular promoters or other transcription-regulating elements.

For instance, in a dual assay, a sample containing two distinct enzymes, such as firefly luciferase and a Renilla luciferase, or any combination of distinct molecules which are capable of being detected by distinct enzyme-mediated luminescence reaction, e.g., a protease and ATP, is assayed. A sample includes a non-cellular sample, e.g., a sample with purified enzymes, an in vitro translation reaction or an in vitro transcription/translation reaction, a cellular (intact) sample, either a prokaryotic or eukaryotic sample, or a cellular lysate. First, an activating (initiating) agent for one of the two enzyme-mediated reactions is added to the sample, in a vessel such as a well in a multi-well plate and the resulting luminescence measured. A specific quench-and-activate reagent is then added to the well so as to selectively quench the first enzyme-mediated reaction, and simultaneously activate the second enzyme-mediated reaction. Or, alternatively, the selective quench reagent and a second light activating reagent specific for the second enzyme-mediated luminescence reaction can be added to the sample sequentially. The luminescence from the second reaction is then measured in the same manner as the first. Optionally, luminescence from the sample may then be quenched by adding a second quench reagent, e.g., a nonselective quench reagent or a selective quench reagent for the second enzyme-mediated reaction to the sample. In this manner, the present invention affords a multiplex luminescence assay capable of measuring two distinct parameters within a single sample. As noted above, one of the enzyme-mediated reactions can act as an internal standard, while the other of the enzyme-mediated reactions may function as a genetic marker or other experimental variable, or alternatively, each reaction can measure a different experimental variable. Moreover, as the skilled will understand, the method of the invention, being an assay method, will usually be carried out with suitable controls or standards (e.g., a sample being analyzed will be analyzed in parallel with solutions with no enzyme and with known concentrations of enzyme) and, with appropriate standards, the method can be adapted to quantitating the concentration of the molecules to be detected in a test sample (i.e., a sample being analyzed).

For example, the traditional assay chemistries used to quantify the activity of beetle (Wood, 1991) and Renilla (Mathews, et al., 1977) luciferases were incompatible. The present invention embodies innovative chemical formulations that meld the Renilla luciferase assay with that of the firefly or click beetle luciferase reaction, thus creating a novel dual luciferase reporter assay.

In compositions of the invention, e.g., those used in methods of the invention, which are aqueous solutions, the substrate is typically present in a concentration of about 0.01 μM to about 2 mM. For firefly luciferase, luciferin saturates at about 0.47 mM in a reagent optimized for maximal light output and at about 1 mM in a reagent optimized for stable signal. For Renilla luciferase, coelenterazine saturates at about 2 μM in a reagent optimized for maximal light output and at about 60 to 100 μM in a reagent optimized for stable signal. In compositions in which ATP is present, the ATP concentration ranges from about 0.01 mM to about 5 mM, preferably about 0.5 mM. When CoA is present in such compositions which are aqueous solutions, the concentration of CoA ranges from about 0.001 mM to about 5 mM, preferably about 0.2 mM to 1 mM. Similarly, the concentration of DTT present is from about 20 mM to about 200 mM, preferably about 20 to 40 mM.

For sequential Renilla luciferase and beetle luciferase assays, the 100% control value for Reporter #1, the Renilla luciferase-mediated luminescent reaction, is determined by quantifying light emission from the reaction prior to addition of the quench reagent(s). The 100% control value for Reporter #2, e.g., a firefly luciferase-mediated luminescent reaction, is determined by quantifying light emission from a reaction which does not contain the quench reagent(s) and does not contain a substrate for Reporter #1.

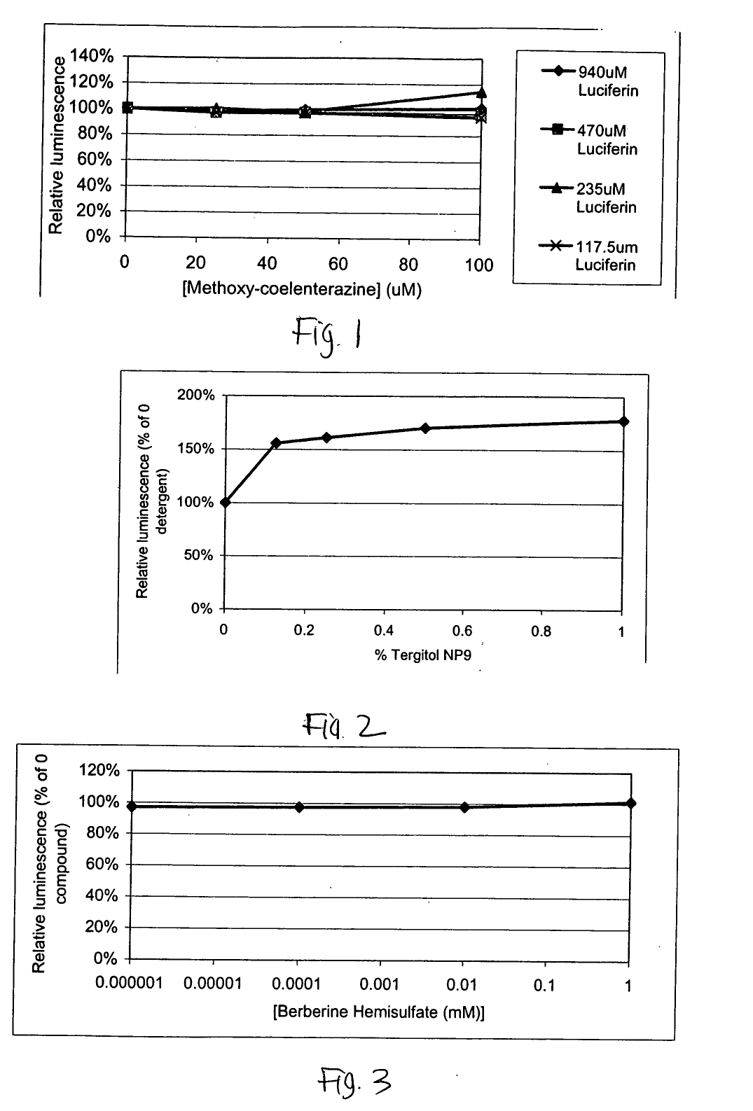

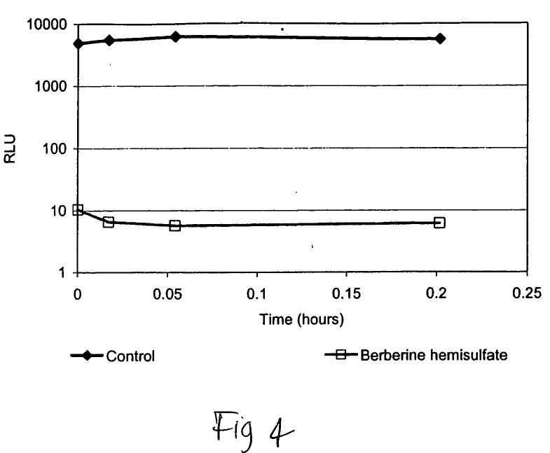

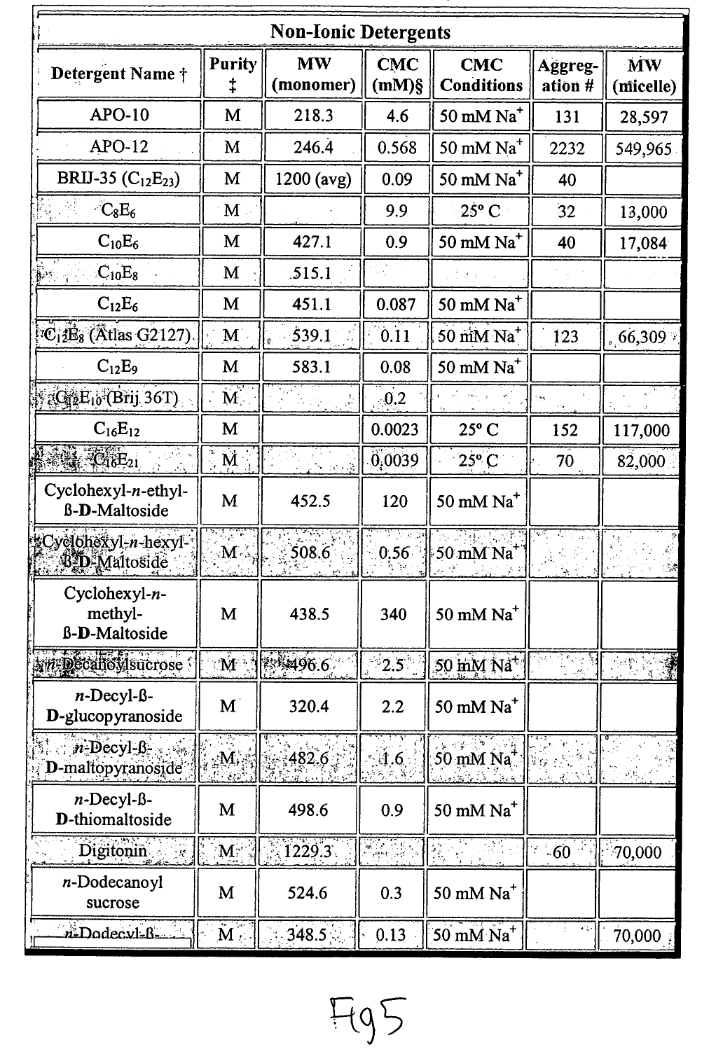

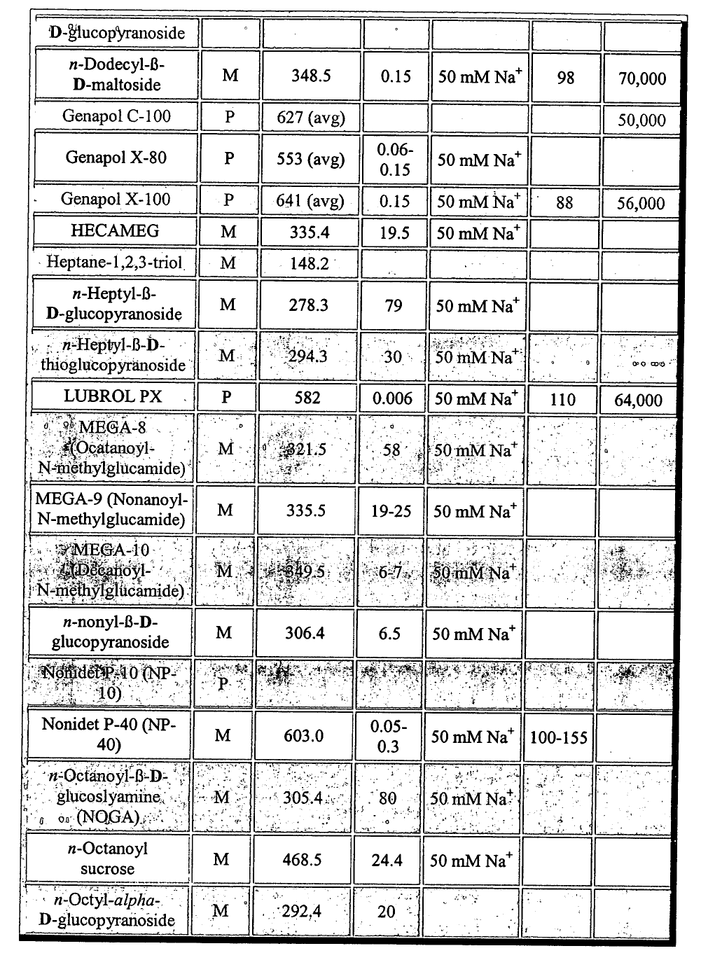

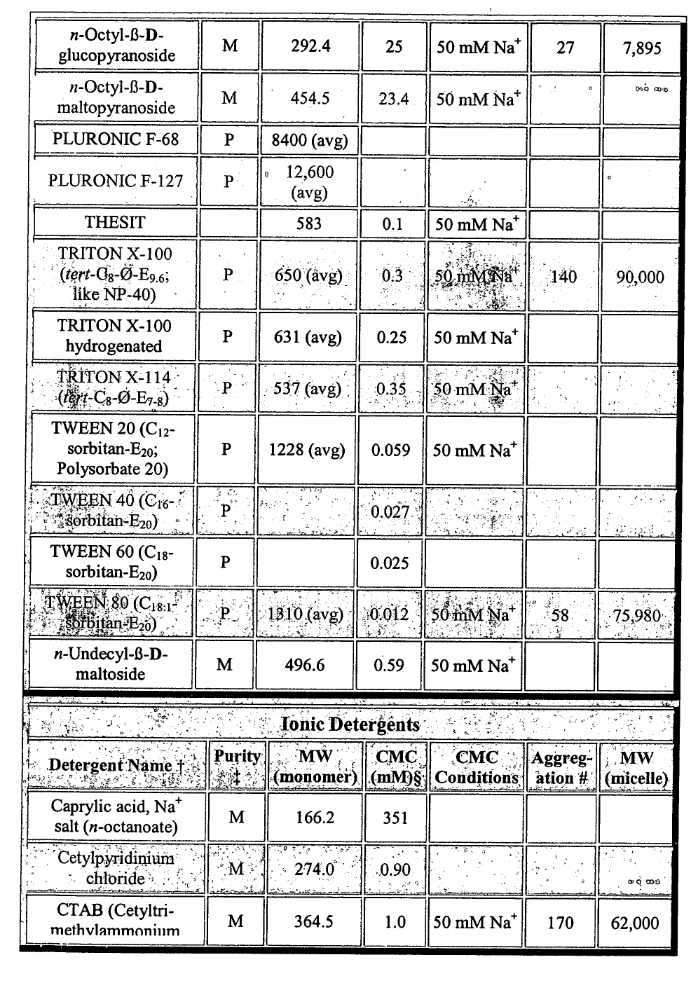

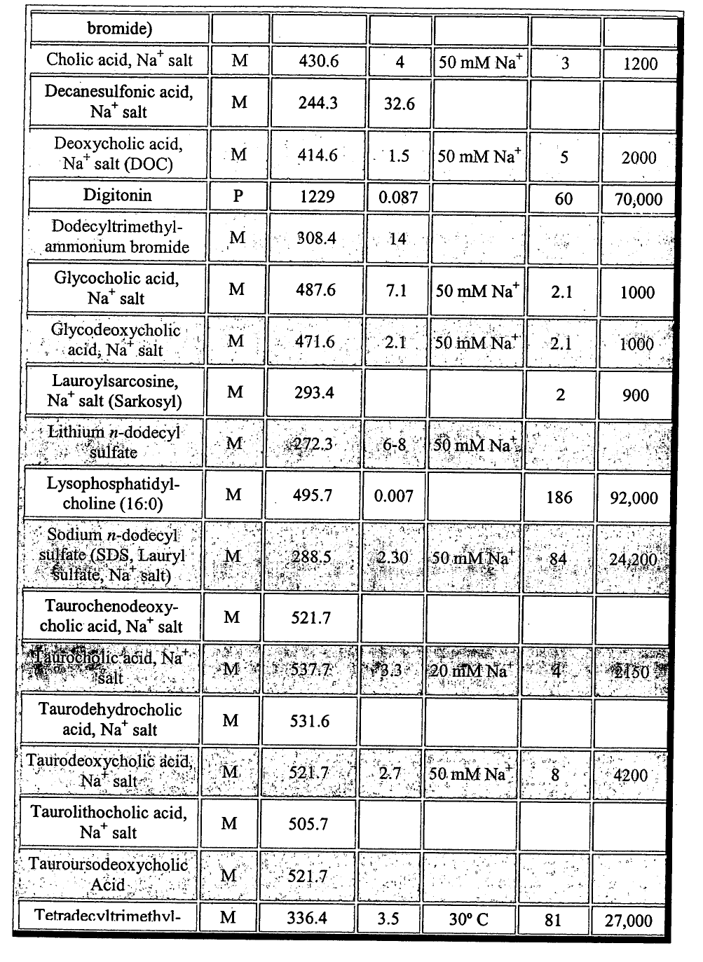

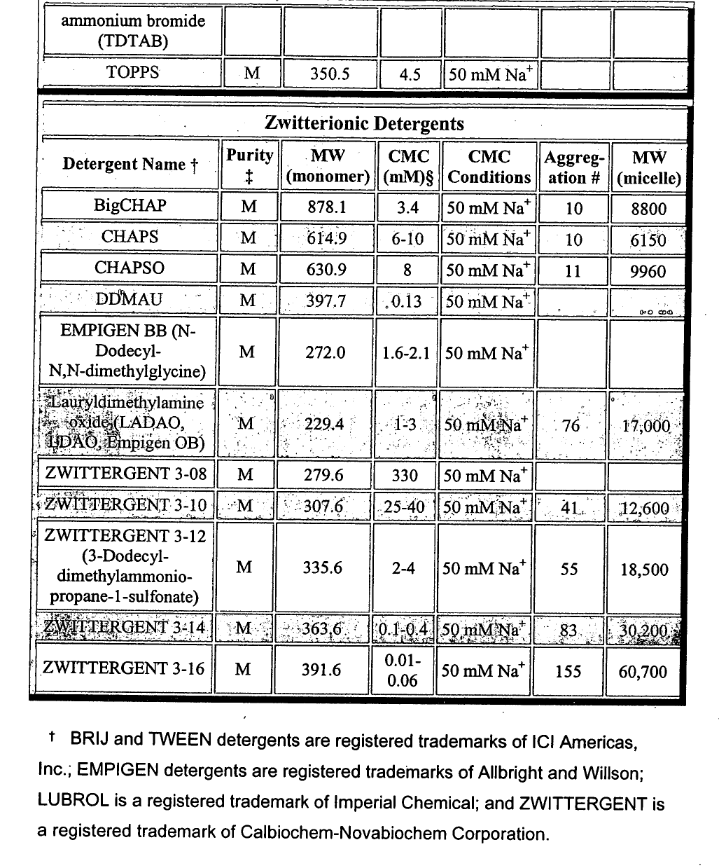

Tables 1-2 and FIG. 4 demonstrate the invention applied to the situation in which a Renilla luciferase-mediated reaction or a horseradish peroxidase-mediated reaction (Reporter #1), is quantified then quenched by the addition of a reagent. In particular, Table 1 demonstrate the invention applied to the situation in which a Renilla luciferase-mediated reaction (Reporter #1) is quantified then selectively quenched by the addition of a composition comprising a substrate analog such as coelenterazine hh methyl ether, a sequestering agent such as Tergitol®, a yellow colored compound such as berberine hemisulfate, or a combination thereof. Those same reagents do not affect the luminescence reaction of firefly luciferase (Reporter #2, see FIGS. 1-3). These examples convincingly demonstrate the unique, integrated nature of the dual luminescent reporter assay. The activity of both luminescent reporter enzymes can be rapidly quantified from within the same sample, contained in a single tube, using the same instrument (Table 1). Thus, the integrated chemistry of the dual assay provides the capability of discriminating the individual luminescent signals from the reaction of two dissimilar luminescent reporter enzymes expressed within a single sample.

As also described herein, white luminometer plates and one or more analytes present in a luminescent enzyme-free luminescence reaction mixture can result in background phosphorescence. To quench this phosphorescence, colored compounds are selected so that the light produced by a luminescence reaction is transmitted, i.e., is detectable, but the light produced by phosphorescence is not transmitted, in the presence of the colored compound. Thus, for red light produced by a red click beetle luciferase, at least one red compound is employed. For green light produced by a green click beetle luciferase, at least one green compound is employed, and for blue light produced by a Renilla luciferase, at least one blue compound is employed. The one or more colored compounds may be added to a reaction mixture prior to addition of a sample having or suspected of having an enzyme which mediates a luminescence reaction, added to the sample prior to the addition of the sample to the reaction mixture, or added when the reaction mixture and sample are combined.

The invention will be further described by the following non-limiting examples.

### EXAMPLE I

### Selective Quench of Renilla Luciferase

The Renilla luciferase luminescent reaction was assessed for its ability to be selectively quenched. Three classes of compounds were tested, a substrate analog of Renilla luciferase, e.g., coelenterazine hh methyl ether, a sequestering agent, e.g., a detergent such as Tergitol NP-9, and/or a yellow colored compound, e.g., berberine hemisulfate.

Materials and Methods

To test the effect of coelenterazine hh methyl ether on a firefly luciferase luminescent reaction, a luciferase reagent was prepared (270 μM coenzyme A (Pharmacia), 530 μM ATP (Pharmacia), 20 mM Tricine pH 7.8 (Fisher), 1 mM magnesium carbonate (Sigma), 0.1 mM ETDA (Sigma), 2.7 mM magnesium sulfate (Sigma), and 33 mM dithiothreitol (Sigma)) with varying concentrations of beetle luciferin (Promega), both above and below the concentration required for luciferase saturation (940 μM, 470 μM, 235 μM and 117.5 μM, saturation occurs at about 470 μM). Coelenterazine hh methyl ether (Promega Biosciences) was solubilized in DMSO and added to the different luciferase reagents at 0 μM, 20 μM, 50 μM and 100 μM. Luminescence from firefly luciferase was measured by adding 20 μl of firefly luciferase (5×10⁻¹⁴ moles/reaction) (Promega Corp.) in 1× Cell Culture Lysis Reagent (Promega Corporation) containing 1 mg/ml bovine serum albumin (BSA) to 100 μl of the luciferase reagents. Luminescence was normalized to the value integrated in the absence of coelenterazine hh methyl ether.

To test the effect of Tergitol® NP-9 on the firefly luciferase luminescent reaction, Luciferase Assay Reagent (Promega Corporation) was prepared according to the manufacturer's instructions. Tergitol NP-9 (Sigma) was titrated into the reagent. Luminescence was integrated after adding 20 μl of firefly luciferase (2.5×10⁻¹⁴ moles/reaction) in 150 mM HEPES pH 7.4 and 1 mg/ml gelatin, to 100 μl of reagent. Luminescence was normalized to the value integrated for no detergent.

To test the effect of berberine hemisulfate on the firefly luciferase luminescent reaction, Steady-Glo® Reagent (Promega Corporation) was prepared according to the manufacturer's instructions. Berberine hemisulfate was solubilized in DMSO and was titrated into the reagent at various concentrations. Firefly luciferase was diluted to approximately 2.2×10⁻¹⁵ moles/reaction in F12 medium (Life Technologies) containing 1 mg/ml BSA (Fisher). Luminescence reactions were initiated by combining 100 μl of Steady-Glo® Reagent and 100 μl of diluted enzyme. Luminescence was normalized to the value integrated for no detergent.

To test the effect of coelenterazine hh methyl ether, Tergitol® NP-9 and/or berberine hemisulfate on Renilla luciferase luminescent reaction, Renilla Luciferase Assay Reagent (Promega Corporation) was prepared according to the manufacturer's instructions. Luciferase Assay Buffer (pt. E152, Promega Corporation) was combined with 1% Tergitol NP-9, 200 μM coelenterazine hh methyl ether, 1 mM berberine hemisulfate, or combinations of the three. Each buffer was added to a vial of Luciferase Assay Substrate (pt. El 51, Promega Corporation) to make Luciferase Assay Reagent (LAR) plus the quenching agent(s). Renilla luciferase (5×10-14 moles/reaction) was prepared in 150 mM HEPES (pH 7.471) plus 1 mg/ml of gelatin. Luminescence was initiated by addition of 20 μl of enzyme solution to 100 μl of Renilla Luciferase Assay Reagent, and measured. Subsequent addition of 100 μl Luciferase Assay Reagent allowed for the Renilla luminescence to be quenched, and the residual luminescence to be measured. Fold quench was calculated as the quotient of the initial Renilla luciferase luminescence divided by the residual Renilla luciferase luminescence.

Results

Each of the tested selective quenching reagents was shown to have little deleterious effect on the firefly luciferase luminescent reaction (FIGS. 1-3). Those same reagents were then tested for their ability to quench Renilla luciferase mediated-luminescence (Table 1). For higher concentrations of coelenterazine hh methyl ether, the addition of certain agents, e.g., a sequestering agent such as Tergitol NP-9 (Sigma), were required to maintain and/or increase solubility. Moreover, quenching by coelenterazine hh methyl ether was increased due to the presence of the sequestering agent.

Yellow dyes were examined for their tendency to absorb the blue light from a Renilla luciferase luminescent reaction without affecting the light output from the firefly reaction. Of the yellow dyes tested, dipyridamole (data not shown) and berberine hemisulfate were shown to be selective quenching reagents for the Renilla luciferase luminescent reaction (for instance, see Table 1 and FIG. 3). For example, dipyridamole at 1 mM was found to quench the Renilla luciferase luminescent reaction by about 35-fold and berberine hemisulfate at 1 mM was found to quench the reaction by about 46-fold to 89-fold.

Although none of the selective quenching agents deleteriously affected the firefly luciferase luminescent reaction their individual and combined effects on the Renilla luciferase luminescent reaction were dramatic.

TABLE 1

| | | Coelenterazine | Berberine | |
|---|---|---|---|---|
| | Detergent | hh methyl ether | Hemisulfate | Fold quench |
| | (Sequestering | (Substrate | (Colored | of Renilla |
| Sample | Agent) | Analog) | Compound) | luciferase |
| 1 | − | − | − | 2.11 |
| 2 | + | − | − | 86.67 |
| 3 | − | + | − | 77.6 |
| 4 | + | + | − | 320 |
| 5 | − | − | + | 46 |
| 6 | + | − | + | 409.5 |
| 7 | − | + | + | 279 |
| 8 | + | + | + | 988 |

### EXAMPLE II

### Use of Selective Quench Reagents for Sequential Luciferase Measurements

Materials and Methods

Renilla Luciferase Assay Reagent (Promega Corporation) was prepared according to manufacturer's instructions. Luciferase Assay Buffer (pt. E152, Promega Corporation) was combined with 1% Tergitol NP-9, 200 μM coelenterazine hh methyl ether, and 1 mM berberine hemisulfate. Luciferase Assay Buffer was added to the Luciferase Assay Substrate (pt. E151, Promega Corporation) to make Luciferase Assay Reagent plus quenching agents. Enzyme stocks for the assay were prepared in 150 mM HEPES (pH 7.471) plus 1 mg/ml of gelatin (for enzyme stability). A stock of Renilla luciferase and firefly luciferase at the final concentrations of about 5×10⁻¹² and 5×10⁻¹⁴ moles/reaction, respectively, as well as a 50:50 mixture of the Renilla and firefly luciferase stocks above were prepared. Luminescence was generated by adding 20 μl of each enzyme stock to 100 μl of Renilla Luciferase Assay Reagent and integrating the luminescence. Subsequent addition of 100 μl Luciferase Assay Reagent allowed for the Renilla luminescence reaction to be quenched and the firefly luminescence to be measured. The firefly luciferase luminescence or the residual Renilla luciferase luminescence was then measured for each of the enzyme samples. The luminescence values for the enzyme sample containing the 50:50 mix of firefly and Renilla luciferases were doubled to normalize enzyme concentration.

TABLE 2

| | | Firefly Luminescence |
|---|---|---|
| | | of Residual Renilla |
| Enzyme Sample | Renilla Luminescence | Luminescence |
| Renilla Luciferase | 283593.3 RLU | 287.3 RLU |
| Firefly Luciferase | 25.3 RLU | 27210.0 RLU |
| Renilla & Firefly | 145579.3 RLU | 13652.7 RLU |
| Luciferase (as | | |
| measured) | | |
| Renilla & Firefly | 291158.6 RLU | 27305.4 RLU |
| Luciferases (normalized | | |
| for enzyme | | |
| concentration) | | |

Results

The data in Table 2 show that a second enzyme, firefly luciferase, can reliably be measured following quench of the first enzymatic reaction, Renilla luciferase reaction, using a combination of the three quench reagents. Thus, the use of a modified Luciferase Assay Reagent to quench the Renilla luminescent reaction permits both Renilla and firefly enzymes to be accurately measured from the same sample.

### EXAMPLE III

### Quenching Light from a Horseradish Peroxidase Luminescence Reaction with a Colored Compound

Materials and Methods

20 μl of 0.044 mg/ml horseradish peroxidase (HRP), prepared in KPO₄, pH 6.5, was added to 100 μl of 50 mM NaHCO₃, 2 μM H₂O₂+/−100 μM berberine hemisulfate. The control reaction did not contain berberine hemisulfate. 100 μl of 10 mM Luminol (Sigma) in 55 mM NaOH was then added to initiate the chemiluminescent reaction and the luminescence was measured on a luminometer. Luminescence was captured at various times after reaction initiation.

Results

As is evident in Table 1, berberine hemisulfate (a yellow compound) can be used to quench the output of light from Renilla luciferase (which emits blue luminescence). An HRP-mediated reaction also can generate blue light. FIG. 4 shows that yellow compounds can be utilized to quench light from an HRP-based reporter system. For example, berberine hemisulfate quenched horseradish peroxidase-dependent chemiluminescence by over 500-fold. Thus, sequential luminescence measurements of multiple reporter proteins can be measured from the same well where one of the reporters is HRP.

### EXAMPLE IV

### Quenching Phosphorescence from Plates or Analytes

The use of white luminometer plates for luminescent reactions often results in background phosphorescence. In phosphorescence, light emitted by an atom or molecule persists after the exciting source is removed. It is similar to fluorescence, but the species is excited to a metastable state from which a transition to the initial state is forbidden. Emission occurs when thermal energy raises the electron to a state from which it can de-excite, resulting in the gradual release of that energy over time in the visible band. Therefore, phosphorescence is temperature-dependent. To quench this phosphorescence, thereby increasing the signal/background ratio, colored compounds were chosen so that the light produced by a particular luciferase would be effectively transmitted but the light from the phosphorescence would not be.

Materials and Methods

Amaranth and benzopurpurin 4B are red compounds and red click beetle luciferase emits red light. Fluorescent brightener 28 is a yellow compound and firefly luciferase emits a yellow-green light.

Stocks of Amaranth (Aldrich, 120561), Benzopurpurin 4B (Aldrich # 22882), and Fluorescence Brightener 28 (Aldrich 475300) were prepared in DMSO (Sigma) at 100 μM. Luminometer plates (96-well) were purchased from Dynex Technologies. The luminometer plates were broken into pieces that would fit into single luminometer tubes (12 mm diameter) purchased from Promega Corporation. All experiments were performed in a lab under normal fluorescent lighting.

The experiment measured the signal/background ratio before and after the addition of colored compounds or DMSO. Luminescence measurements were taken from the empty luminescent tube in each experiment to quantitate background. A piece of white luminescent plate, 100 μl of Bright-Glo™ Reagent prepared according to the manufacturer's instructions (Promega Corporation), and 100 μl of Glo Lysis Buffer (Promega Corporation) were placed into the luminescent tube, and the luminescence was again measured. This measurement captured the phosphorescence emitted from the luminometer plate in a commercial firefly luciferase reagent. 2 μl of DMSO or one of the dyes in DMSO were then added to the tube, the sample was mixed, and the luminescence was measured a third time. This measurement captured the amount of luminescence emitted through the now-colored reagent or the reagent containing the DMSO carrier. Finally, 2 μl of luciferase was added to the tube, the sample was mixed, and the luminescence measured a final time. All luminescence measurements were 10 second integrations after 2 second delay.

The firefly luciferase was QuantiLum® luciferase from Promega Corporation at a concentration of 1.4×10⁻⁵ mg/ml in Glo Lysis Buffer containing 1 mg/ml porcine gelatin (Sigma Chemical). Red click beetle luciferase was obtained from a cell lysate made with Glo Lysis Buffer from CHO cells transiently transfected with red click beetle luciferase. Although the absolute luciferase concentration in this sample is unknown, the improvement in signal/background can be evaluated with any amount of luciferase that generates luminescence above the background.

The background subtracted luminescence from the luciferase sample was divided by the background-subtracted luminescence of the reagent+plate piece sample to calculate the signal/background ratio of the phosphorescence. The background-subtracted luminescence from the luciferase sample was divided by the background-subtracted luminescence of DMSO- or dye-added sample to calculate the signal/background ratio of the DMSO or dye sample. The signal/background improvement then is the ratio in the presence of DMSO or dye divided by the ratio of the phosphorescence then minus 1, and is expressed as a percent.

TABLE 3
Signal/Background Ratios
Red Click Beetle

| Phosphorescence S/B | Add DMSO, S/B | S/B Improvement |
|---|---|---|
| 2290 | 3018 | 32% |
| 1420 | 1453 | 2% |
| Phosphorescence S/B | Amaranth | S/B Improvement |
| 756 | 3056 | 304% |
| 583 | 8010 | 1274% |
| 1402 | 4843 | 245% |
| Phosphorescence S/B | Benzopurpurin 4B | S/B Improvement |
| 1121 | −6537 | NA |
| 2029 | −9277 | NA |
| 1144 | 52624 | 4500% |

Firefly

| Phosphorescence S/B | Add DMSO, S/B | S/B Improvement |
|---|---|---|
| 989 | 906 | −8% |
| Phosphorescence S/B | Fluorescence Brightener | S/B Improvement |
| 899 | 4855 | 440% |
| 1985 | −19193 | NA |

Results

As shown in Table 3, colored compounds, such as red compounds for a red click beetle luciferase-mediated reaction, and yellow compounds for a firefly luciferase-mediated reaction, when added to the respective reactions, improved the signal to background ratio.

The negative numbers in Table 3 indicate that the samples containing dye have luminescence lower than the background measured for the tube alone. The signal/background improvement then cannot be calculated for those samples because the value is infinite.

Thus, colored compounds may, in a homogeneous system, be present in a reagent added to cells, prior to measuring luminescence. For a nonhomogeneous system, the colored compound may be present in a lysing reagent which is added to cells, after which a reagent for the reaction is added and then luminescence is measured. Alternatively, a lysing reagent may be added to cells, after which a reagent for the reaction which includes the colored compound is added, and then luminescence is measured.

### REFERENCES

Annaert et al., Pharmaceut. Res., 14, 492 (1997).

Blaise et al., BioTechniques, 16, 932 (1994).

Bronstein, et al., Anal-Biochem., 219, 169 (1994).

Bronstein, et al., Bioluminescence and Chemiluminescence: Current Status. (eds. P. E. Stanley and L. J. Kricka) John Wiley & Sons, Inc. pp. 73-82 (1991).

Denburg et al., Archives of Biochemistry and Biophysics, 134, 381 (1969).

Denburg et al., Archives of Biochemistry and Biophysics, 141, 668 (1970).

Flanagan et al., J. Virology, 65, 769 (1991).

Inoue et al., Tetrahedron Letters, 31, 2685 (1977).

Jain et al., BioTechniques, 12, 681 (1992).

Kobatake et al., Bioluminescence and Chemiluminescence (ed. A. A. Szalay, et al.) John Wiley & Sons, Chichester, pp. 337-341 (1993).

Kondepudi et al., Poster abstract #725, presented at annual meeting of the American Society of Cell Biologist, Dec. 10-14, 1994, San Francisco, Calif.

Leckie et al., BioTechniques, 17, 52 (1994).

Lee et al., Archives of Biochemistry and Biophysics, 141, 38-52 (1970).

Mathews et al., Biochemistry, 16, 85 (1977).

Redden et al., Int. J. Pharm., 180, 151 (1999).

Schaap et al., Clinical Chemistry, 35, 1863 (1989).

Schram, Bioluminescence and Chemiluminescence: Current Status. (eds. P. E. Stanley and L. J. Kricka) John Wiley & Sons, Inc., pp. 407-412 (1991).

Thompson et al., Gene, 103, 171 (1991).

Thorp et al., Methods in Enzymology, 133, 331 (1986).

Tsien, Nature, 290, 527 (1981).

U.S. Pat. No. 5,831,102.

Ward, Chemi- and Bioluminescence (ed. John Burr) Marcel Dekker, Inc., New York, pp. 321-358 (1985).

Wood, Curr. Op. Biotech., 6, 50 (1995).

Wood, in Bioluminescence & Chemiluminescence: Current Status. (eds. Stanley, P. E., and Kricka, J.) John Wiley & Sons, Chichester. pp.543-546 (1991).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A method of assaying an enzyme-mediated luminescence reaction comprising:
(a) detecting or determining luminescence energy produced by at least one first enzyme-mediated luminescence reaction which is not a beetle luciferase-mediated reaction; and
(b) introducing a composition capable of selectively quenching the first enzyme-mediated luminescence reaction and initiating a second enzyme-mediated luminescence reaction distinct from the first enzyme-mediated luminescence reaction, wherein the composition comprises at least one selective quench reagent for the first enzyme-mediated luminescence reaction; and
(c) detecting or determining luminescence energy produced by the second enzyme-mediated luminescence reaction.

2. A method of assaying an enzyme-mediated luminescence reaction comprising:
(a) detecting or determining luminescence energy produced by at least one first enzyme-mediated luminescence reaction; and
(b) quenching photon emission from the first enzyme-mediated luminescence reaction by introducing a composition comprising a colored compound to the luminescence reaction which compound is a selective quench reagent.

3. A method of assaying an enzyme-mediated luminescence reaction comprising:
(a) detecting or determining luminescence energy produced by at least one first enzyme-mediated luminescence reaction which is not a beetle luciferase-mediated reaction; and
(b) quenching photon emission from the first enzyme-mediated luminescence reaction by introducing a composition comprising at least one selective quench reagent to the luminescence reaction;
(c) introducing a composition capable of initiating a second enzyme-mediated luminescence reaction distinct from the first enzyme-mediated luminescence reaction; and
(d) detecting or determining luminescence energy produced by the second enzyme-mediated luminescence reaction.

4. The method according to claim 2 in which the composition further comprises reagents capable of initiating a second enzyme-mediated luminescence reaction distinct from the first enzyme-mediated luminescence reaction; and
(c) detecting or determining luminescence energy produced by the second enzyme-mediated luminescence reaction.

5. The method according to claim 1 or 3 wherein at least one selective quench reagent is a substrate analog inhibitor for the first enzyme.

6. The method according to claim 1 or 3 wherein at least one selective quench reagent is a sequestering agent.

7. The method according to claim 6 wherein the sequestering agent sequesters a substrate for the first enzyme but not the second enzyme.

8. The method according to claim 6 wherein the sequestering agent is a nonionic detergent.

9. The method according to claim 6 wherein the sequestering agent is a crown ether, glycol, or cyclodextran.

10. The method according to claim 1 or 3 wherein at least one selective quench reagent is a colored compound.

11. The method according to claim 10 wherein the colored compound quenches blue, green or red light.

12. The method according to claim 1 or 3 wherein in step (a), a luciferase-mediated luminescence reaction is detected or determined.

13. The method according to claim 12 wherein the luciferase-mediated luminescence reaction is mediated by an anthozoan luciferase or a functional equivalent thereof.

14. The method according to claim 12 wherein in step (b), the first enzyme-mediated reaction is quenched with a nonionic detergent which is not Triton® X-100 or Tween® 20, a substrate analog inhibitor which is a protected coelenterazine, a yellow compound, or a combination thereof.

15. The method according to claim 14 wherein the luciferase-mediated luminescence reaction is mediated by Renilla reniformis (sea pansy) luciferase or a functional equivalent thereof.

16. The method according to claim 2 wherein the colored compound quenches blue, green or red light.

17. The method according to claim 2 or 4 wherein in step (a), a luciferase-mediated luminescence reaction is detected or determined.

18. The method according to claim 17 wherein the luciferase-mediated luminescence reaction is mediated by an anthozoan luciferase or a functional equivalent thereof.

19. The method according to claim 18 wherein the luciferase-mediated luminescence reaction is mediated by Renilla reniformis (sea pansy) luciferase or a functional equivalent thereof.

20. The method according to claim 1, 3 or 4 wherein the second enzyme-mediated luminescence reaction is mediated by an anthozoan luciferase or a functional equivalent thereof.

21. The method according to claim 20 wherein the second enzyme-mediated luminescence reaction is mediated by Renilla reniformis (sea pansy) luciferase or a functional equivalent thereof.

22. The method according to claim 1, 3 or 4 wherein the second enzyme-mediated luminescence reaction is mediated by a luciferase.

23. The method according to claim 22 wherein the second enzyme-mediated luminescence reaction is mediated by Photinus pyralis (North American firefly) luciferase, Pyrophorous plagiophthalamus luciferase, or a functional equivalent thereof.

24. The method according to claim 1, 3 or 4 wherein one of the enzyme-mediated luminescence reactions detects the presence or amount of a substrate, enzyme or cofactor.

25. The method according to claim 1, 2, 3 or 4 wherein in step (a), a peroxidase-mediated luminescence reaction is detected or determined.

26. The method according to claim 25 wherein a horseradish peroxidase-mediated luminescence reaction is detected or determined.

27. The method according to claim 1, 2, 3 or 4 wherein in step (a), a phosphatase-mediated luminescence reaction is detected or determined.

28. The method according to claim 27 wherein alkaline phosphatase-mediated luminescence reaction is detected or determined.

29. The method according to claim 1, 3 or 4 wherein the second enzyme-mediated luminescence reaction is a peroxidase-mediated luminescence reaction.

30. The method according to claim 29 wherein the second enzyme-mediated luminescence reaction is a horseradish peroxidase-mediated luminescence reaction.

31. The method according to claim 1, 3 or 4 wherein the second enzyme-mediated luminescence reaction is a phosphatase-mediated luminescence reaction.

32. The method according to claim 31 wherein the second enzyme-mediated luminescence reaction is an alkaline phosphatase-mediated luminescence reaction.

33. The method according to claim 1, 3 or 4 wherein in step (a), a first luciferase-mediated luminescence reaction is detected or determined; and the second enzyme-mediated luminescence reaction is a second and distinct luciferase-mediated luminescence reaction.

34. The method according to claim 33 wherein in step (a), the first enzyme-mediated luminescence reaction is mediated by an anthozoan luciferase or a functional equivalent thereof; and the second enzyme-mediated luminescence reaction is mediated by a beetle luciferase or a functional equivalent thereof.

35. The method according to claim 34 wherein the second enzyme-mediated luminescence reaction is mediated by a Photinus pyralis or a Pyrophorus plagiophthalamus luciferase.

36. The method according to claim 34 wherein in step (a), the first enzyme-mediated luminescence reaction is mediated by Renilla reniformis luciferase.

37. The method according to claim 2 wherein the reaction detects the presence or amount of a substrate, enzyme or cofactor.

38. The method according to claim 1, 3 or 4 further comprising:
subsequent to detecting or determining luminescence energy produced by the second enzyme-mediated luminescence reaction, quenching the second enzyme-mediated luminescence reaction by introducing a composition comprising at least one second quench reagent capable of quenching the second enzyme-mediated luminescence reaction.

39. The method of claim 38 wherein the at least one second quench reagent is capable of selectively quenching the second enzyme-mediated reaction.

40. The method of claim 1, 2 or 3 wherein the selective quench reagent quenches the first enzyme-mediated luminescence reaction by at least 35-fold.

41. The method of claim 1, 2 or 3 wherein more than one selective quench reagent is present in the composition.

42. The method of claim 41 wherein the selective quench reagents quench the first enzyme-mediated luminescence reaction by at least 100-fold.

43. An enzyme-mediated luminescence reaction assay kit comprising:
at least one functional enzyme substrate for a molecule to be detected by the enzyme-mediated luminescence reaction, wherein the substrate is not a beetle luciferase substrate;
a suitable first container, the at least one functional enzyme substrate disposed therein;
a composition comprising at least one selective quench reagent which is a substrate analog inhibitor for the enzyme which mediates the luminescence reaction, a colored compound, or a nonionic detergent which is not Triton® X-100 or Tween® 20;
a suitable second container, the composition disposed therein; and
instructions for use.

44. An enzyme-mediated luminescence reaction assay kit comprising:
at least one functional enzyme substrate for a molecule to be detected by the enzyme-mediated luminescence reaction;
a suitable first container, the at least one functional enzyme substrate disposed therein;
a composition comprising at least one selective quench reagent for an anthozoan luciferase;
a suitable second container, the composition disposed therein; and
instructions for use.

45. The kit according to claim 43 or 44 wherein the selective quench reagent is a substrate analog inhibitor which is a protected coelenterazine.

46. The kit according to claim 43 or 44 wherein the selective quench reagent is a crown ether, glycol, or cyclodextran.

47. The kit according to claim 44 wherein the selective quench reagent is a nonionic detergent which is not Triton® X-100 or Tween® 20.

48. The kit according to claim 43 or 44 wherein the selective quench reagent is a yellow colored compound.

49. A dual reporter enzyme-mediated luminescence reaction assay kit comprising:
a first functional enzyme substrate for a molecule to be detected by a first enzyme-mediated luminescence reaction;
a suitable first container, the first functional enzyme substrate disposed therein;
a quench-and-activate composition comprising at least one selective quench reagent for an enzyme which mediates the first luminescence reaction and a second and distinct functional enzyme substrate corresponding to a second and distinct enzyme-mediated luminescence reaction, wherein the enzyme which mediates the first luminescence reaction is not a beetle luciferase;
a suitable second container, the quench-and-activate composition disposed therein; and
instructions for use.

50. A dual reporter enzyme-mediated luminescence reaction assay kit comprising:
a first functional enzyme substrate for a molecule to be detected by a first enzyme-mediated luminescence reaction, wherein the substrate is not a substrate for a beetle luciferase;
a suitable first container, the first functional enzyme substrate disposed therein;
a quench-and-activate composition comprising at least one selective quench reagents and a second and distinct functional enzyme substrate corresponding to a second and distinct enzyme-mediated luminescence reaction;
a suitable second container, the quench-and-activate composition disposed therein; and
instructions for use.

51. The kit according to claim 49 or 50 wherein the first functional enzyme substrate, and the second and distinct functional enzyme substrate, are luciferase substrates.

52. The kit according to claim 49 or 50 which comprises a nonionic detergent which is not Triton® X-100 or Tween® 20, a substrate analog inhibitor which is a protected coelenterazine, a yellow compound, or a combination thereof.

53. The kit according to claim 49 or 50 wherein the sequestering agent is a crown ether, glycol, or cyclodextran.

54. The kit according to claim 49 or 50 further comprising:
a second quench reagent capable of quenching photon emission from the second and distinct enzyme-mediated reaction; and
a suitable third container, the second quench reagent disposed therein.

55. A method of assaying an enzyme-mediated luminescence reaction comprising:
(a) detecting or determining luminescence energy produced by at least one first enzyme-mediated luminescence reaction; and
(b) quenching photon emission from the first enzyme-mediated luminescence reaction by introducing at least one quench reagent to the luminescence reaction, wherein the quench reagent comprises a nonionic detergent that is not Triton® X-100 or Tween® 20, a substrate analog inhibitor for an anthozoan luciferase, a colored compound, or a combination thereof.

56. A method of assaying an enzyme-mediated luminescence reaction comprising:
(a) detecting or determining luminescence energy produced by at least one first enzyme-mediated luminescence reaction; and
(b) quenching the first enzyme-mediated luminescence reaction by introducing a composition comprising at least one quench reagent to the luminescence reaction, wherein the quench reagent comprises a nonionic detergent that is not Triton® X-100 or Tween® 20, a substrate analog inhibitor for an anthozoan luciferase, a colored compound, or a combination thereof.

57. A method to reduce or inhibit analyte-independent or analyte-dependent phosphorescence in an enzyme-mediated luminescence reaction, comprising:
(a) contacting a sample comprising an enzyme that mediates a luminescence reaction with a reaction mixture for the enzyme comprising a colored compound, which mixture does not comprise the enzyme, wherein the color of the compound is substantially the same as the light emitted in the luminescence reaction; and
(b) detecting or determining luminescence energy.

58. A method to reduce or inhibit analyte-independent or analyte-dependent phosphorescence in an enzyme-mediated luminescence reaction, comprising:
(a) contacting a sample comprising an enzyme that mediates a luminescence reaction and a colored compound with a reaction mixture for the enzyme, wherein the color of the compound is substantially the same as the light emitted in the luminescence reaction; and
(b) detecting or determining luminescence energy.

59. The method according to claim 57 or 58 further comprising detecting or determining luminescence energy in the reaction mixture prior to contacting the mixture with the sample.

60. The method according to claim 57 or 58 wherein the compound is a red, yellow, blue or green colored compound.

61. The method according to claim 57 or 58 wherein the enzyme is an anthozoan luciferase.

62. The method according to claim 57 or 58 wherein the enzyme is a beetle luciferase.

63. An enzyme-mediated luminescence reaction assay kit comprising:
at least one functional enzyme substrate for a molecule to be detected by the enzyme-mediated luminescence reaction;
a suitable first container, the at least one functional enzyme substrate disposed therein;
at least one colored compound;
a suitable second container, the at least one colored compound disposed therein; and
instructions for use,
wherein the color of the at least one compound is substantially the same as the light emitted by the enzyme-mediated luminescence reaction.

64. An enzyme-mediated luminescence reaction assay kit comprising:
at least one colored compound and at least one functional enzyme substrate for a molecule to be detected by the enzyme-mediated luminescence reaction;
a suitable first container, the at least one colored compound and the at least one functional enzyme substrate disposed therein; and
instructions for use,
wherein the color of the at least one compound is substantially the same as the light emitted by the enzyme-mediated luminescence reaction.

65. An enzyme-mediated luminescence reaction assay kit comprising:
a quench-and-activate composition comprising at least one selective quench reagent for an enzyme which mediates a luminescence reaction and a functional enzyme substrate for a molecule to be detected by a second and distinct enzyme-mediated luminescence reaction, wherein the enzyme which mediates the first luminescence reaction is not a beetle luciferase;
a suitable container, the quench-and-activate composition disposed therein; and
instructions for use.

66. The kit of claim 65 wherein the second enzyme-mediated luminescence reaction is a beetle luciferase-mediated luminescence reaction.

67. The kit of claim 65 wherein the first enzyme-mediated luminescence reaction is an anthozoan luciferase-mediated luminescence reaction.

68. The kit of claim 65 wherein the selective quench reagent is a nonionic detergent that is not Triton® X-100 or Tween® 20, a substrate analog inhibitor for an anthozoan luciferase, a colored compound, or a combination thereof.

69. A method of assaying an enzyme-mediated luminescence reaction comprising:
(a) detecting or determining luminescence energy produced by at least one first enzyme-mediated luminescence reaction which is not a beetle luciferase-mediated reaction; and
(b) introducing a composition capable of selectively quenching the first enzyme-mediated luminescence reaction and initiating a second enzyme-mediated luminescence reaction distinct from the first enzyme-mediated luminescence reaction, wherein the composition comprises at least one selective quench reagent which is a substrate analog inhibitor for the first enzyme, a nonionic detergent that is not Triton® X-100 or Tween® 20, or a colored compound; and
(c) detecting or determining luminescence energy produced by the second enzyme-mediated luminescence reaction.
